(19) United States
(12) Patent Application Publication
(10) Pub. No.: US 2026/0096878 A1
(43) Pub. Date: Apr. 9, 2026

(54) SYSTEMS AND METHODS FOR TREATING A DEFECTIVE CARDIAC VALVE HAVING ANTIMIGRATION FEATURES

(71) Applicant: CroiValve Ltd., Dublin (IE)

(72) Inventors: Vincent HULK, Dublin (IE); Gavin KENNY, Galway (IE); Pascal LAUNOIS, Dublin (IE); Ricky UNADKAT, Dublin (IE); Amit TUBISHEVITZ, Tel Aviv (IL)

(73) Assignee: CroiValve Ltd., Dublin (IE)

(21) Appl. No.: 19/333,867

(22) Filed: Sep. 19, 2025

Related U.S. Application Data
(60) Provisional application No. 63/704,452, filed on Oct. 7, 2024.

(51) Int. Cl. A61F 2/24

(52) U.S. Cl. CPC A61F 2/2412; A61F 2/2427; A61F 2220/0008; A61F 2230/0006; A61F 2230/001; A61F 2250/0018; A61F 2250/0039

(57) ABSTRACT

Apparatus and methods for repairing a cardiac valve, e.g., a tricuspid valve, are provided. The apparatus may include a prosthetic device coupled to an elongated support coupled to a stent configured to be implanted within a vessel, e.g., the superior vena cava, to suspend and maintain the prosthetic device within the cardiac valve. The support may include a proximal, delivery portion detachably coupled, in a delivery state, to a distal, implantable portion coupled to the prosthetic device. The prosthetic device may be formed of biocompatible material coupled to a frame, and may have prosthetic leaflets that allows blood to flow therethrough in one direction during diastole, but prevents blood regurgitation during systole. The stent further may include features configured to resist migration of the stent within the vessel, as well as to prevent in-folding of the stent during deployment.

### CROSS-REFERENCE TO RELATED APPLICATIONS

This application claims the benefit of priority to U.S. Provisional Patent Appl. No. 63/704,452, filed Oct. 7, 2024, the entire contents of which are incorporated herein by reference.

### TECHNICAL FIELD

This technology generally relates to systems having antimigration features and methods for performing transcatheter or minimally invasive repair of a defective cardiac valve, such as the tricuspid, mitral, pulmonary, and aortic valves.

### BACKGROUND

The human heart has four major valves which moderate and direct blood flow in the cardiovascular system. These valves serve critical functions in assuring a unidirectional flow of an adequate blood supply through the cardiovascular system. The mitral valve and aortic valve control the flow of oxygen-rich blood from the lungs to the body. The mitral valve lies between the left atrium and left ventricle, while the aortic valve is situated between the left ventricle and the aorta. Together, the mitral and aortic valves ensure that oxygen-rich blood received from the lungs is ejected into systemic circulation. The tricuspid and pulmonary valves control the flow of oxygen-depleted blood from the body to the lungs. The tricuspid valve lies between the right atrium and right ventricle, while the pulmonary valve is situated between the right ventricle and the pulmonary artery. Together the tricuspid and pulmonary valves ensure unidirectional flow of oxygen-depleted blood received from the right atrium towards the lungs.

Heart valves are passive structures composed of leaflets that open and close in response to differential pressures on either side of the valve. The aortic, pulmonary, and tricuspid valves have three leaflets, while the mitral valve has only two leaflets. Dysfunction of the cardiac valves is common and can have profound clinical consequences. Regurgitation occurs when the valve leaflets do not meet, or “coapt” correctly, thus causing blood to leak backwards through the valve each time the heart pumps. Failure of the valves to prevent regurgitation leads to an increase in the pressure of blood in the lungs or liver and reduces forward blood flow, causing the heart to pump more blood to compensate for the loss of pressure. Such degradation may result in serious cardiovascular compromise or even death. Valvular dysfunction either results from a defect in the valve leaflet or supporting structure, or dilation of the fibrous ring supporting the valve. These factors lead to poor coaptation of valve leaflets, allowing blood to travel in the wrong direction.

Previously known medical treatments to address diseased valves generally involve either repairing the diseased native valve or replacing the native valve with a mechanical or biological valve prosthesis. Previously-known valve prostheses have some disadvantages, such as the need for long-term maintenance with blood thinners, the risk of clot formation, limited durability, etc. Accordingly, valve repair, when possible, usually is preferable to valve replacement. However, most dysfunctional valves are too diseased to be repaired using previously known methods and apparatus. Accordingly, a need exists for a prosthesis capable of assisting heart valve function that enables treatment of a larger patient population, while reducing the need to fully supplant the native heart valve.

For many years, the standard treatment for such valve dysfunction called for surgical repair or replacement of the valve during open-heart surgery, a procedure conducted under general anesthesia. An incision is made through the patient's sternum (sternotomy), and the heart is accessed and stopped while blood flow is rerouted through a heart-lung bypass machine. When replacing the valve, the native valve is excised and replaced with either a mechanical or biological prosthesis. However, these surgeries are prone to many complications and long hospital stays for recuperation.

More recently, transvascular techniques have been developed for introducing and implanting a replacement valve, using a flexible catheter in a manner less invasive than open-heart surgery. In such techniques, a replacement valve is mounted in a compressed state at the end of a flexible catheter and advanced through the blood vessel of a patient until the prosthetic valve reaches the implantation site. The valve then is expanded to its functional size at the site of the defective native valve, usually by inflating a balloon within where the valve has been mounted. By expanding the prosthetic valve, the native valve leaflets are generally pushed aside and rendered ineffective. Examples of such devices and techniques, wherein the native valve is replaced in its entirety by a substitute tissue valve, are described, for example, in U.S. Pat. Nos. 6,582,462 and 6,168,614 to Andersen.

Prostheses have been produced and used for over sixty years to treat cardiac disorders. They have been made from a variety of materials, both biological and artificial. Mechanical or artificial valves generally are made from non-biological materials, such as plastics or metals. Such materials, while durable, are prone to blood clotting and thrombus formation, which in turn increases the risk of embolization and stroke or ischemia. Anticoagulants may be taken to prevent blood clotting that may result in thromboembolic complications and catastrophic heart failure, however, such anti-clotting medication may complicate a patient's health due to the increased risk of hemorrhage.

In contrast, “bio-prosthetic” valves are constructed with prosthetic leaflets made of natural tissue, such as bovine, equine or porcine pericardial tissue, which functions very similarly to the leaflets of the natural human heart valve by imitating the natural action of the heart valve leaflets, coapting between adjacent tissue junctions known as commissures. The main advantage of valves made from natural tissue is they are not as prone to blood clots and do not absolutely require lifelong systemic anticoagulation.

In recent years, bio-prosthetic valves have been constructed by integrating prosthetic leaflets made from natural tissue into a stent-like supporting frame, which provides a dimensionally stable support structure for the prosthetic leaflets. In more advanced prosthetic heart valve designs, besides providing dimensionally stable support structure for the prosthetic leaflets, the stent-like supporting frame also imparts a certain degree of controlled flexibility, thereby reducing stress on the prosthetic leaflet tissue during valve opening and closure and extending the lifetime of the prosthetic leaflets. In most designs, the stent-like supporting frame is covered with a biocompatible cloth (usually a polyester material such as Dacron™ or polytetrafluoroethylene (PTFE)) that provides sewing attachment points for the prosthetic leaflet commissures and prosthetic leaflets themselves. Alternatively, a cloth-covered suture ring may be attached to the stent-like supporting frame, providing a site for sewing the valve structure in position within the patient's heart during a surgical valve replacement procedure.

While iterative improvements have been made on surgical bio-prosthetic valves over the last several decades, existing bio-prosthetic valves still have drawbacks. In most designs, the bio-prosthetic valve is implanted as a replacement for the native valve, filling the entire space the native valve had occupied. One drawback to this procedure is the mismatch in size and mass between opposing surfaces of the stent-like supporting frame. The mismatch is often due to the variability in the shapes and mechanical characteristics of the stent-like supporting frame. For prosthetic valves with balloon-expandable stent-like supporting frames, the recoil of the supporting frames post-balloon-inflation may lead to perivalvular leaks around the circumference of the prosthetic valve and potential slippage and migration of the valve post-implantation. Another risk associated with prosthetic valves having balloon-expandable supporting frames is potential damage to the prosthetic leaflets of the prosthesis during implantation, when the prosthetic leaflets may be compressed between the balloon and the supporting frame. For prosthetic valves with self-expanding stent-like supporting frames, mismatch may arise due to the deformation/movement of the supporting frame, e.g., slight deformation of the frame into a less than circular shape during normal cardiac movement. Such mismatch may lead to instability among components of a prosthetic valve, resulting in perivalvular leaks and uneven stress distribution in the prosthetic leaflets, resulting in accelerated wear of the valve.

Some innovation has addressed these problems by augmenting, rather than replacing, the native valve. The simplest of these devices is a plug suspended across the center of the valve that allows the native leaflets to coapt against the plug body to block regurgitation, as described in U.S. Pat. No. 7,854,762 to Speziali. Though the plug design helps to prevent regurgitation, the major drawback is that it also blocks some of the blood flow during diastole. Improved prostheses are described in U.S. Pat. Nos. 10,383,729 and 10,682,231 to Quinn, U.S. Pat. No. 10,952,854 to Heneghan et al., and U.S. Pat. No. 11,219,525 to Vesely et al., the entire contents of each of which are incorporated herein by reference.

It would be desirable to further enhance designs to, for example, allow easier delivery of a prosthetic device to a cardiac valve, provide a robust structure that ensures integrity of an implanted prosthetic including its prosthetic leaflets, improve coaptation of the device with the native leaflets to reduce regurgitation.

It would further be desirable to provide an improved anchoring system for maintaining the prosthetic device at the cardiac valve while resisting migration of the anchoring system within a blood vessel coupled to the heart.

### SUMMARY

Provided herein are improved heart valve repair apparatus and methods that, for example, allow more reliable anchoring of the prosthetic device at the native cardiac valve, provide robust structure, and minimize regurgitation. The apparatus and methods may be optimized for use in treating cardiac valve regurgitation when the native leaflets of the cardiac valve do not coapt correctly, thus causing blood to leak backwards through the valve as the heart pumps. Advantageously, apparatus may be configured for implantation at a cardiac valve within a blood flow path such that the native leaflets abut the apparatus during the portion of the cardiac cycle when the cardiac valve attempts to close, thereby enhancing native leaflet coaptation and minimizing regurgitation.

In accordance with one aspect, a system is provided for implanting a therapeutic heart valve device at a native heart valve (e.g., tricuspid, mitral, pulmonary, or aortic valve) of a patient's heart. The system may include a prosthetic device (e.g., a prosthetic valve) that is implanted at the native heart valve, a support coupled to the prosthetic device, and an actuator coupled to the support. The support may include a delivery portion that is used for delivery and an implantable portion that remains coupled to the prosthetic device after delivery and stays implanted with the prosthetic device. During delivery, the delivery portion is attached to the implantable portion and, after suitable placement of the prosthetic device at the native heart valve, the portions are detached and the delivery portion is removed from the patient. For example, the support may have an elongated shaft with a proximal, delivery portion and a distal, implantable portion that is structured to maintain the prosthetic device at the native heart valve.

The actuator may be actuated to cause the components of the distal, implantable portion to lock together, and cause the proximal, delivery portion to detach from the distal, implantable portion at an area (e.g., the detachment area within a blood vessel coupled to the heart such as the inferior vena cava or superior vena cava) responsive to actuation such that the proximal, delivery portion may be removed from the patient while the distal, implantable portion remains implanted within the patient. For example, a clinician may actuate one or more knobs, sliders, buttons, or the like on an actuator, e.g., one or more handles, coupled to the support. The one or more knobs, sliders, buttons, or the like on the actuator may be the same for locking and detachment or may be different. For example, a knob(s) and/or button(s) may be moved in a first direction on the actuator to lock and moved in a second direction (e.g., opposite direction) to detach. The distal, implantable portion of the support may then remain implanted with the prosthetic device to anchor the prosthetic device at a suitable position within the native heart valve.

The support may also permit steering of the prosthetic device during delivery for suitable positioning for implantation. The elongated rail of the support is expected to provide enhanced steering although alternative or additional mechanisms may be used such as pull wire steering. The support further allows for extension and telescoping to increase and/or decrease the length of support for suitable positioning of the prosthetic device at the native cardiac valve. For example, the body support catheter may be capable of telescoping to move the prosthetic device into the suitable position within the native cardiac valve. The support may include an elongated rail having an elongated rail distal portion at the distal, implantable portion of the elongated shaft and an elongated rail proximal portion at the proximal, delivery portion of the elongated shaft. The elongated rail distal portion may be attached to the elongated rail proximal portion during delivery and detached at the detachment area by the actuator for implantation of the elongated rail distal portion.

In accordance with another aspect, a device for maintaining a prosthetic heart valve device having a support at a native heart valve of a patient's heart is provided. The device may comprise a stent tube having a lumen configured to receive the support therethrough, and a stent coupled to the stent tube and configured to transition between a collapsed, delivery state and an expanded, deployed state to anchor the support to a blood vessel coupled to the heart such that the prosthetic heart valve device coupled to the support is positioned at the native heart valve of the patient's heart. The stent may comprise a proximal region, a distal region, and a tapered profile such that a cross-sectional area of the stent increases in a direction from the proximal region towards the distal region. The device further may comprise at least one antimigration clement disposed on the stent and configured to resist migration of the stent in a distal direction when the stent is in the expanded, deployed state within the blood vessel.

The stent may comprise a plurality of circumferentially-extending struts selectively interconnected by a plurality of longitudinal struts. Further, the plurality of circumferentially-extending struts may comprise an alternating pattern of valleys and apexes, the valleys facing in a proximal direction and the apexes facing in the distal direction. The plurality of longitudinal struts may be configured to only connect and extend between selected valleys of adjacent circumferentially-extending struts of the plurality of circumferentially-extending struts, such that the apexes of the plurality of circumferentially-extending struts are unsupported within each cell of the stent defined by a pair of adjacent circumferentially-extending struts of the plurality of circumferentially-extending struts and a pair of adjacent longitudinal struts of the plurality of longitudinal struts.

The at least one antimigration element may comprise the unsupported apexes of the plurality of circumferentially-extending struts, such that, upon radial compression of the stent, the unsupported apexes of the plurality of circumferentially-extending struts are configured to expand radially outwardly in the distal direction at a first angle relative to an outer surface of the stent to interact with an inner wall of the blood vessel and resist migration of the stent in the distal direction. Further, at least one of the unsupported apexes of the plurality of circumferentially-extending struts may be configured to, upon radial compression of the stent, expand radially outwardly in the distal direction at a second angle relative to the outer surface of the stent to interact with the inner wall of the blood vessel and resist migration of the stent in the distal direction, wherein the second angle is greater than the first angle. In addition, the unsupported apexes of the plurality of circumferentially-extending struts configured to expand radially outwardly in the distal direction at the second angle may be disposed in an alternating manner along a circumference of the stent.

Moreover, the unsupported apexes of the plurality of circumferentially-extending struts may be configured to fold radially inward within a sheath to facilitate recapture of the stent as the sheath is advanced over the stent in the distal direction. Additionally, at least one cell of the stent may comprise a local open cell geometry defined by a pair of adjacent longitudinal struts of the plurality of longitudinal struts and a pair of adjacent circumferentially-extending struts of the plurality of circumferentially-extending struts comprising a pair of valleys within the local open cell geometry that are not connected via a longitudinal strut, the local open cell geometry configured to improve flexibility and conformability of the stent within the blood vessel. For example, the at least one cell comprising the local open cell geometry may be disposed in an alternating manner along a circumference of the stent. In addition, in the expanded, deployed state within the blood vessel, the stent may comprise a bow-tie or hourglass shaped profile due to the at least one cell of the stent comprising the local open cell geometry. At least one longitudinal strut of the plurality of longitudinal struts may be configured to extend along an entire length from a valley of a distal-most circumferentially-extending strut of the plurality of circumferentially-extending struts to a valley of a proximal-most circumferentially-extending strut of the plurality of circumferentially-extending struts, adjacent the stent tube.

In some embodiments, the at least one antimigration element may comprise at least one friction pad coupled to at least one longitudinal strut of the plurality of longitudinal struts, such that distally facing edges of the at least one friction pad may be configured to provide additional contact against an inner wall of the blood vessel when the stent is in the expanded, deployed state within the blood vessel to thereby resist migration of the stent in the distal direction. Moreover, at least a portion of an outer surface of the at least one friction pad may comprise a textured surface configured to provide additional friction force against the inner wall of the blood vessel when the stent is in the expanded, deployed state within the blood vessel to thereby resist migration of the stent in the distal direction. Additionally, the at least one friction pad may comprise at least one raised portion configured to be radially elevated from the at least one longitudinal strut via at least one ramped portion. Further, the at least one friction pad may comprise at least one opening disposed on at least one of the at least one raised portion or the at least one ramped portion, the at least one opening configured to provide additional distally facing edges configured to provide additional contact against the inner wall of the blood vessel when the stent is in the expanded, deployed state within the blood vessel to thereby resist migration of the stent in the distal direction.

The at least one friction pad further may comprise a track extending along at least the at least one raised portion, the track configured to reduce impact of stiffness of the at least one longitudinal strut such that the at least one friction pad does not impact conformability of the stent to the blood vessel. The at least one longitudinal strut may extend continuously along the at least one friction pad. Moreover, at least one raised portion may be configured to be radially elevated from the at least one longitudinal strut via a proximal ramped portion extending from a proximal flat portion extending laterally from the at least one longitudinal strut, and via a distal ramped portion extending from a distal flat portion extending laterally from the at least one longitudinal strut. Alternatively, the at least one longitudinal strut may be discontinuous along the at least one friction pad. Additionally, the track may comprise a geometry having additional distally facing edges configured to provide additional contact against the inner wall of the blood vessel when the stent is in the expanded, deployed state within the blood vessel to thereby resist migration of the stent in the distal direction.

In addition, the at least one friction pad may comprise at least one gap extending laterally inward from at least one lateral side of the at least one friction pad, the at least one gap configured to provide additional distally facing edges configured to provide additional contact against the inner wall of the blood vessel when the stent is in the expanded, deployed state within the blood vessel to thereby resist migration of the stent in the distal direction. The stent further may comprise at least one additional circumferentially-extending strut extending distally from the distal region of the stent, the at least one additional circumferentially-extending strut configured to facilitate even deployment of the stent from a sheath. Additionally, or alternatively, at least one circumferentially-extending strut of the plurality of circumferentially-extending struts may comprise a stiffness that increases in a direction towards a connection of the stent to the stent tube to thereby resist bending of the stent along the connection of the stent to the stent tube.

Moreover, the stent may be configured to be implanted within a superior vena cava, and the at least one antimigration element may be disposed on the proximal region of the stent such that, when the stent is in the expanded, deployed state within the superior vena cava, the at least one antimigration element is positioned cranial to the patient's pulmonary artery and/or aorta. Additionally, or alternatively, the stent may be configured to be implanted within a superior vena cava, and the at least one antimigration element may be selectively disposed at radial positions along the stent such that, when the stent is in the expanded, deployed state within the superior vena cava, the at least one antimigration element is positioned away from the patient's aorta. The stent may be configured to be implanted within a superior vena cava such that a proximal end of the stent is positioned close to, but does not completely cross, a confluence of the patient's brachiocephalic vein.

In some embodiments, the at least one antimigration element may comprise at least one short flare configured to transition between a collapsed configuration and an expanded configuration where the at least one short flare is angled relative to the outer surface of the stent in the distal direction to interact with the inner wall of the blood vessel and resist migration of the stent in the distal direction. For example, the at least one short flare may be configured to be pivotally coupled to at least one longitudinal strut of the plurality of longitudinal struts. Further, the at least one short flare may comprise a bone-shaped profile configured to provide additional flexibility and reduce a risk of sheath delamination upon stent deployment. Alternatively, the at least one short flare may comprise a straight, linear profile. Moreover, a distal end of the at least one short flare may comprise a rounded tip configured to reduce a risk of vessel perforation and sheath delamination upon stent deployment.

In addition, the stent may comprise a distal atraumatic portion comprising a closed cell structure extending from a distal end of the distal region of the stent, the distal atraumatic portion comprising at least one thin circumferentially-extending strut that is more flexible than the plurality circumferentially-extending struts of the stent to provide a soft, gradual transition of interaction between an inner wall of the blood vessel and the stent. Additionally, or alternatively, the stent may comprise a proximal atraumatic portion comprising a closed cell structure extending from a proximal end of the proximal region of the stent, the proximal atraumatic portion comprising at least one thin circumferentially-extending strut that is more flexible than the plurality circumferentially-extending struts of the stent to provide a soft, gradual transition of interaction between an inner wall of the blood vessel and the stent. Further, the stent may be configured to be implanted within a superior vena cava, and the at least one antimigration clement may comprise the proximal atraumatic portion such that, in the expanded, deployed state, the proximal atraumatic portion is configured to flare radially outward and conform to a confluence of the patient's left and right brachiocephalic veins coupled to the superior vena cava to resist migration of the stent in the distal direction without crossing the confluence.

The stent may comprise an expandable wire frame having variable stiffness along a length of the stent. For example, the expandable wire frame may comprise a decoupling section extending between and connecting a proximal-most circumferentially-extending strut of the distal region of the stent and a distal-most circumferentially-extending strut of the proximal region of the stent, the decoupling section configured to limit transmission of force from the distal region to the proximal region. The proximal and distal regions of the stent may comprise a stiffness that is greater than a stiffness of the decoupling section of the stent, and the distal region and the decoupling section may be more deflectable than the proximal region. In addition, the distal region of the stent may be configured to be coupled to the stent tube, and the proximal region of the stent may be configured to contact an inner wall of the blood vessel in the expanded, deployed state to anchor the support to the blood vessel. Accordingly, the decoupling section may be configured to limit transmission of force from the distal region to the proximal region to resist migration of the stent in the distal direction.

In some embodiments, the decoupling section may comprise a plurality of wavy struts extending between the proximal-most circumferentially-extending strut of the distal region of the stent and the distal-most circumferentially-extending strut of the proximal region of the stent. For example, the plurality of wavy struts may comprise an S-shape. Additionally, or alternatively, the decoupling section may comprise a plurality of suture attachments configured to connect the proximal-most circumferentially-extending strut of the distal region of the stent and the distal-most circumferentially-extending strut of the proximal region of the stent. Moreover, the distal region of the stent may be fixedly coupled to a distal portion of the stent tube via a fixed connection, and the proximal region of the stent may be fixedly coupled to a sliding ring, the sliding ring slidably coupled to a proximal portion of the stent tube such that movement of the sliding ring along the stent tube relative to the fixed connection causes a middle region of the stent between the proximal and distal regions to transition between a contracted configuration and an expanded configuration. The sliding ring may be configured to be selectively locked to the stent tube to maintain a desired degree of expansion of the middle region of the stent.

Moreover, the stent tube may comprise at least one tilt control rib extending radially outward from an outer surface of the stent tube in a direction toward a central axis of the stent. The at least one tilt control rib may be sized and shaped to push the stent tube against an inner wall of a sheath when the stent is in the collapsed, delivery state within the sheath to thereby prevent in-folding of the stent during deployment from the sheath. Alternatively, the device further may comprise a balloon catheter configured to be disposed between the stent tube and the stent when the stent is in the collapsed, delivery state within a sheath. The balloon catheter may comprise a balloon configured to be inflated during deployment of the stent from the sheath to push the stent tube away from a medial wall of the blood vessel to thereby prevent in-folding of the stent during deployment from the sheath. Additionally, or alternatively, the device further may comprise a sheath comprising a slit extending proximally from a distal end of the sheath, the sheath configured to receive the stent in the collapsed, delivery state such that the stent tube is disposed oppositely from the slit. Accordingly, during deployment of the stent from the sheath, the slit may facilitate deployment of the portion of the stent opposite the stent tube towards a medial wall of the blood vessel to thereby push the stent tube away from the medial wall of the blood vessel and prevent in-folding of the stent during deployment from the sheath. Additionally, or alternatively, the stent may comprise a stent spine comprising a proximal portion affixed to the stent tube and a flexible distal portion unaffixed to the stent tube. Accordingly, during deployment of the stent from a sheath, the flexible distal portion of the stent spine may be configured to lift radially away from the stent tube to thereby prevent in-folding of the stent during deployment from the sheath.

In accordance with another aspect, a device for maintaining a prosthetic heart valve device having a support at a native heart valve of a patient's heart is provided. The device may comprise a stent tube having a lumen configured to receive the support therethrough, and a stent coupled to the stent tube and configured to transition between a collapsed, delivery state and an expanded, deployed state to anchor the support to a blood vessel coupled to the heart such that the prosthetic heart valve device coupled to the support is positioned at the native heart valve of the patient's heart. A distal region of the stent may be fixedly coupled to a distal portion of the stent tube via a fixed connection, and a proximal region of the stent may be fixedly coupled to a sliding ring, the sliding ring slidably coupled to a proximal portion of the stent tube such that movement of the sliding ring along the stent tube relative to the fixed connection causes a middle region of the stent between the proximal and distal regions to transition between a contracted configuration and an expanded configuration. In addition, the sliding ring may be configured to be selectively locked to the stent tube to maintain a desired degree of expansion of the middle region of the stent.

In accordance with another aspect, a device for maintaining a prosthetic heart valve device having a support at a native heart valve of a patient's heart is provided. The device may comprise a stent tube having a lumen configured to receive the support therethrough, a stent coupled to the stent tube and configured to transition between a collapsed, delivery state and an expanded, deployed state to anchor the support to a blood vessel coupled to the heart such that the prosthetic heart valve device coupled to the support is positioned at the native heart valve of the patient's heart, and an in-folding prevention element configured to prevent in-folding of the stent during deployment from the sheath. For example, the in-folding prevention element may comprise at least one tilt control rib extending radially outward from an outer surface of the stent tube in a direction toward a central axis of the stent. The at least one tilt control rib may be sized and shaped to push the stent tube against an inner wall of a sheath when the stent is in the collapsed, delivery state within the sheath to thereby prevent in-folding of the stent during deployment from the sheath. Alternatively, the in-folding prevention element may comprise a balloon catheter configured to be disposed between the stent tube and the stent when the stent is in the collapsed, delivery state within the sheath. The balloon catheter may comprise a balloon configured to be inflated during deployment of the stent from the sheath to push the stent tube away from a medial wall of the blood vessel to thereby prevent in-folding of the stent during deployment from the sheath. Additionally, or alternatively, the in-folding prevention element may comprise a sheath comprising a slit extending proximally from a distal end of the sheath, the sheath configured to receive the stent in the collapsed, delivery state such that the stent tube is disposed oppositely from the slit. Accordingly, during deployment of the stent from the sheath, the slit may facilitate deployment of the portion of the stent opposite the stent tube towards a medial wall of the blood vessel to thereby push the stent tube away from the medial wall of the blood vessel and prevent in-folding of the stent during deployment from the sheath. In some embodiments, the stent may comprise a stent spine comprising a proximal portion affixed to the stent tube and a flexible distal portion unaffixed to the stent tube, and the in-folding prevention element may comprise the flexible distal portion of the stent spine configured to lift radially away from the stent tube during deployment from the sheath to thereby prevent in-folding of the stent during deployment from the sheath.

### BRIEF DESCRIPTION OF THE DRAWINGS

FIG. 1 is a perspective view of an exemplary heart valve therapeutic device for repairing a defective heart valve.

FIGS. 2A and 2B are perspective views of an exemplary prosthetic device of the heart valve therapeutic device of FIG. 1.

FIG. 2C is a cross-sectional view of the prosthetic device of FIGS. 2A and 2B.

FIGS. 2D and 2E are perspective views of an exemplary frame of the prosthetic device of FIGS. 2A-2C.

FIG. 2F is a close up view of an exemplary step up of the frame of the prosthetic device of FIGS. 2A-2C.

FIG. 3A illustrates the implantable portion of the heart valve therapeutic device of FIG. 1 implanted at a native heart valve.

FIGS. 3B and 3C illustrate the heart valve therapeutic device at a native heart valve in a closed and open configuration, respectively.

FIG. 4 illustrates exemplary detachable supports constructed in accordance with some embodiments.

FIG. 5 is a perspective view of an exemplary anchor and anchor tube of the detachable support of FIG. 4.

FIG. 6A is a side view of exemplary components of the anchor tube of the detachable support, and FIG. 6B is a side view of the anchor tube of the detachable support in an engaged position without the anchor tube sleeve shown.

FIGS. 7A to 7C illustrate the anchor tube in an engaged configuration suitable for delivering the prosthetic device to the cardiac valve and a disengaged configuration where the distal, implantable portion remains implanted and the proximal, delivery portion can be removed from the patient.

FIG. 8 is a perspective view of an exemplary stent constructed in accordance with some embodiments.

FIGS. 9A to 9D illustrate various configurations of frames for the stent of the anchor.

FIG. 10 is a perspective view of an alternative exemplary anchor and anchor tube constructed in accordance with some embodiments.

FIGS. 11A to 11H are views of an exemplary method for introducing the prosthetic device of the heart valve therapeutic device across a native heart valve for implantation.

FIGS. 12A to 12C illustrate the implantable portion of the heart valve therapeutic device of FIG. 1 maintained at a native heart valve via the support.

FIGS. 13A and 13B illustrate another alternative exemplary anchor and anchor tube.

FIGS. 14A and 14B illustrate the bow tie shape of the anchor of FIGS. 13A and 13B when disposed within a superior vena cava in accordance with some embodiments.

FIG. 15A illustrates an exemplary anchor having short flares disposed on the stent of the anchor, and FIGS. 15B to 15D illustrate various configurations of the short flares.

FIGS. 15E and 15F illustrate the anchor of FIG. 15A disposed within a superior vena cava to maintain the heart valve therapeutic device at a native heart valve via the support.

FIGS. 16A to 16C illustrate another alternative exemplary anchor having flared cells.

FIG. 17A illustrates an exemplary anchor having friction pads disposed on the stent of the anchor, and FIGS. 17B to 17E illustrate various configurations of the friction pads.

FIG. 18 illustrates an exemplary anchor having a flared cranial end region.

FIG. 19 is a schematic illustrating an exemplary anchor having a high deflection decoupling section.

FIGS. 20A to 20C illustrate an exemplary anchor having a decoupling section formed by wavy struts of the frame of the stent of the anchor.

FIG. 21 illustrates an alternative exemplary anchor having a decoupling section formed by suture attachments of the frame of the stent of the anchor.

FIGS. 22A and 22B illustrate a selectively expandable anchor constructed in accordance with some embodiments.

FIGS. 23A to 23C illustrate potential in-folding during deployment of an expandable anchor from a delivery sheath.

FIGS. 24A and 24B illustrate an exemplary expandable anchor having tilt control ribs to facilitate deployment of the expandable anchor in accordance with some embodiments.

FIGS. 25A to 25C illustrate deployment of an expandable anchor via an exemplary balloon catheter in accordance with some embodiments.

FIGS. 26A and 26B illustrate an exemplary delivery sheath having a slit to facilitate deployment of an expandable anchor constructed in accordance with some embodiments.

FIG. 27 illustrates an exemplary expandable anchor having variable stiffness to facilitate deployment of the expandable anchor in accordance with some embodiments.

FIGS. 28A and 28B illustrate an exemplary expandable anchor having a liftable stent spine portion to facilitate deployment of the expandable anchor in accordance with some embodiments.

FIG. 29 illustrates select implantable components of an alternative exemplary heart valve therapeutic device having two anchors.

### DETAILED DESCRIPTION

Embodiments of this technology are directed to exemplary systems and methods for reducing cardiac valve regurgitation. Provided herein is a prosthetic device that may contain a prosthetic coaptation body to be positioned at a native cardiac valve. The prosthetic device may be suspended across the native heart valve by a support. For example, the support may be coupled to the prosthetic coaptation body and extend out of the heart into an adjacent blood vessel coupled to the heart (e.g., superior vena cava, inferior vena cava). The support may be coupled to the blood vessel with an anchor that preferably is expandable and has a stent structure. In some examples, the support is structured to suspend the prosthetic coaptation body in the native valve in a free-standing manner without anchoring to cardiac tissue, thereby minimizing damage to the heart. The prosthetic coaptation body may be formed from a frame (e.g., metal frame such as Nitinol) that is at least partially covered by a skirt made from biocompatible material, and also includes prosthetic leaflets. The frame, biocompatible material, and prosthetic leaflets may together form a conduit through which blood flows when the prosthetic leaflets open during the cardiac cycle.

The design of the prosthetic device improves coaptation with the native heart valve leaflets and allows for a more reliable delivery. The prosthetic device may be implanted percutaneously via a blood vessel, e.g., the jugular vein, femoral vein, femoral artery, for the treatment of a defective cardiac valve, e.g., tricuspid, mitral, pulmonary, or aortic valve. In one example, the prosthetic device may be used to treat symptomatic primary or functional (secondary) tricuspid regurgitation. For example, the prosthetic device may be positioned between the native tricuspid valve leaflets to restore the valve function without altering the native anatomy or obstructing flow during diastole and held in place by an anchor system deployed in an anchor site, e.g., within the heart and/or within a blood vessel coupled to the heart such as the superior vena cava (SVC).

The frame may be designed with predefined kink points or collapsible/expandable features to allow the conduit to be compressed into a delivery sheath without being damaged, and to more reliably expand upon delivery. The frame may have a proximal ring and a distal ring, as well as an inner ring coupled to the proximal ring via a plurality of skirt anchors to which the prosthetic valve leaflets may be attached. One or more of the rings may exhibit a scallop, sinusoidal, zig-zag shape or otherwise oscillating pattern in the expanded state to further improve the compression and expansion of the frame. The skirt of the prosthetic coaptation body may join the proximal ring to the distal ring to improve coaptation of the native valve against the skirt. The prosthetic coaptation body may be coupled to the support by a plurality of tethers that may be formed of shape-memory material such as Nitinol. The tethers may be rigid or stiff and hold the prosthetic coaptation body in position more accurately than tensile wires.

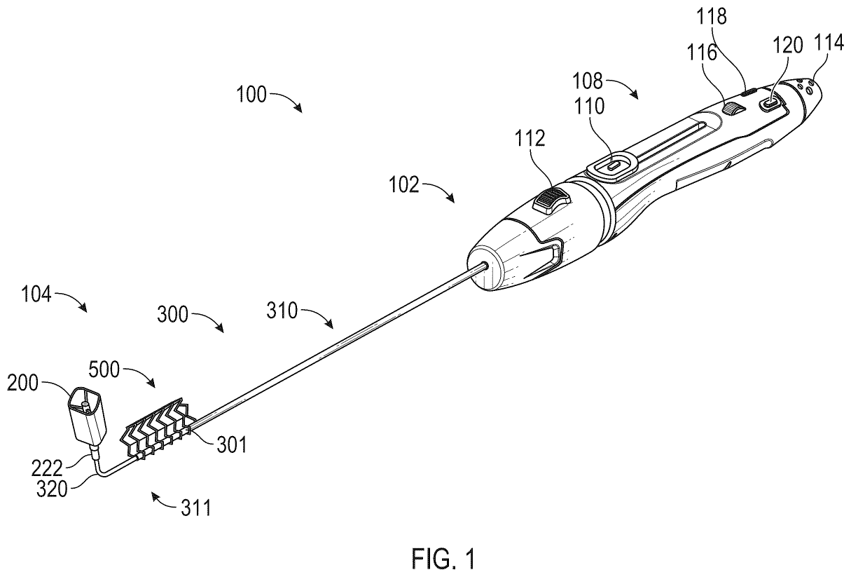

Referring to FIG. 1, an illustrative embodiment of exemplary heart valve therapeutic device 100 is described. Illustratively, heart valve therapeutic device 100 is designed for repairing a defective tricuspid valve. As will be understood by a person having ordinary skill in the art, heart valve therapeutic device 100 may be readily adapted for other cardiac valves such as the mitral valve, aortic valve, or pulmonary valve. Heart valve therapeutic device 100 may be constructed as described in Int'l Patent Appl. Pub. No. WO 2024/161309 A1, Int'l Patent Appl. Pub. No. WO 2023/026113 A1, and U.S. Pat. No. 11,219,525, the entire contents of each of which are incorporated by reference herein.

As illustrated in FIG. 1, heart valve therapeutic device 100 may include prosthetic device 200 coupled to support 300 at distal region 104 of heart valve therapeutic device 100, as well as actuator 108 at proximal region 102 of heart valve therapeutic device 100. Actuator 108 may include one or more handles configured to be manipulated by a clinician to deliver the system for implantation. Support 300 may include an elongated shaft including proximal, delivery portion 310 and distal, implantable portion 311, such that proximal, delivery portion 310 is removeably coupled to distal, implantable portion 311 at detachment area 301, and distal, implantable portion 311 is coupled to prosthetic device 200 at valve connection area 222. Heart valve therapeutic device 100 is structured to deliver prosthetic device 200 to a damaged native heart valve for an acute or chronic treatment, and certain components of heart valve therapeutic device 100 such as distal, implantable portion 311 of support 300 may be designed to be fully implanted long-term for the chronic treatment.

Distal, implantable portion 311 of support 300 further may include anchor 500. Anchor 500 may be formed of a stent structure and is preferably collapsible in a contracted, delivery state and expandable to an expanded, deployed state to anchor the prosthetic device at the native cardiac valve. For example, anchor 500 may contact the inner wall of a blood vessel (e.g., the SVC or IVC) to anchor distal, implantable portion 311 of support 300 intraluminally, thereby anchoring prosthetic device 200 in a free-standing, suspended manner in the native heart valve. As shown in FIG. 1, anchor 500 may be positioned on distal, implantable portion 311 adjacent to detachment area 301 of support 300. In some examples, detachment area 301 is located within anchor 500 such that the proximal end of anchor 500 provides the proximal-most position of the implantable portion of the device.

Actuator 108 is designed to be held and manipulated by a clinician and may include one or more interfaces such as interfaces 110, 112, 114, 116, 118, and 120. As illustrated, actuator 108 may be coupled to the proximal region support 300 and interfaces 110, 112, 114, 116, 118, and 120 may each be coupled to corresponding components of support 300 such that actuation of the interfaces cause movements described herein for delivery and implantation of prosthetic device 200. Interfaces 110, 112, 114, 116, 118, and 120 may be buttons, sliders, knobs, or the like that are actuated to deliver prosthetic device 200, manipulate support 300 for suitable implantation, lock distal components of distal, implantable portion 311 together, and/or to detach proximal, delivery portion 310 from distal, implantable portion 311. Accordingly, responsive to actuation of the interfaces of actuator 108, prosthetic device 200 may be manipulated for suitable positioning within the target native heart valve, the distal components of distal, implantable portion 311 may be locked together, and proximal, delivery portion 310 may be detached from distal, implantable portion 311. For example, interface 110 may be operatively coupled to the shaping catheter for making extension adjustments to extend prosthetic device 200 into implantation position. Interface 112 may be operatively coupled to the elongated rail for adjusting the angle of the rail for positing the prosthetic device 200 at the appropriate angle relative to the native heart valve. Interface 114 may be operatively coupled to the body support catheter for telescoping adjustments to extend or retract prosthetic device 200 to the native heart valve. Interface 116 may be operatively coupled to a first lock to lock distal, implantable components of the support together for implantation. For example, interface 116 may be operatively coupled to the body support catheter pusher for actuating the body support catheter lock. Interface 118 may be operatively coupled to a second lock to lock different distal, implantable components of the support together for implantation, such as locking to the anchor system. For example, interface 118 may be operatively coupled to the shaping catheter pusher for actuating the shaping catheter lock. Interface 120 may be operatively coupled to the anchor tube sleeve for disengaging the anchor tube.

In some configurations, an interface, e.g., interface 116, 118, may be moved distally along handle to cause portions of support 300 to move distally in a corresponding manner to facilitate locking of the distal components of distal, implantable portion 311 to secure the components in the implantable, locked position suitable for short-term (acute) or long-term (chronic) implantation of prosthetic device 200 at the native cardiac valve, as explained in detail below. Further, the same or different interface(s) may be moved proximally along the handle to cause detachment of the proximal, delivery portion 310 from distal, implantable portion 311 such that proximal, delivery portion 310 may be removed from the patient while distal, implantable portion 311 remains implanted, as explained in detail below. Interfaces 110, 112, 114, 116, 118, and 120 may be manually operated or controlled remotely using motorized controls, and actuator 108 may be actuated to reattach proximal, delivery portion 310 to distal, implantable portion 311 post-implantation in a follow-up procedure to permit adjustments after implantation of prosthetic device 200.

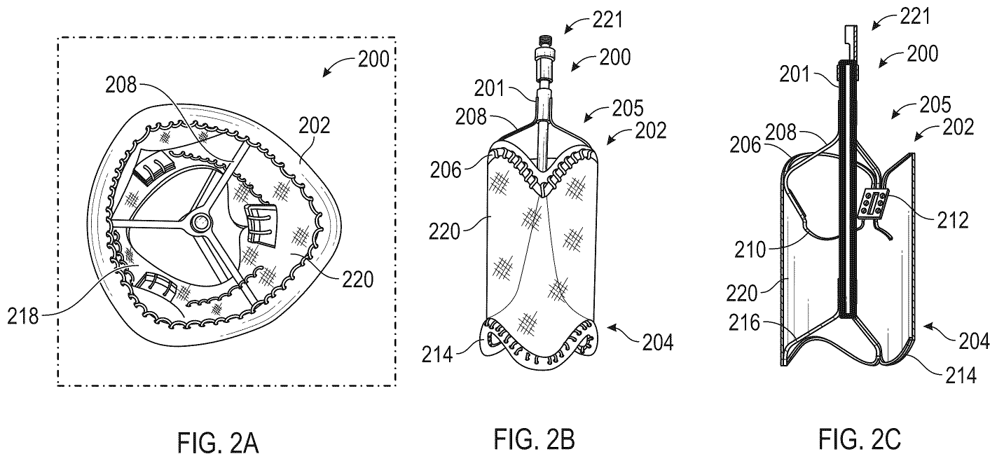

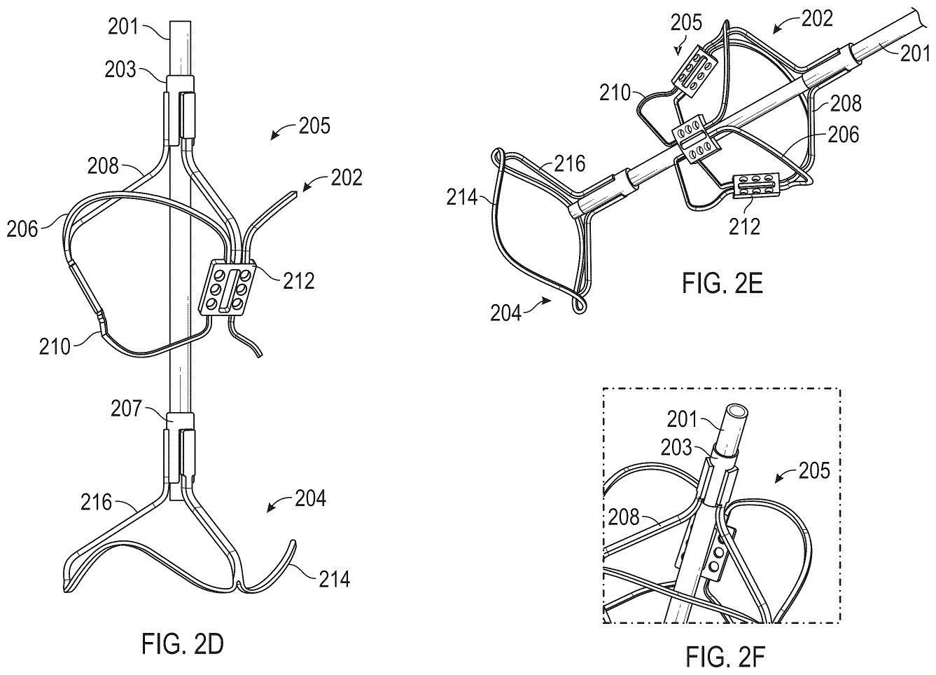

Prosthetic device 200 may be a prosthetic coaptation body 200, as illustrated, that includes a prosthetic valve structured to enhance the function of the native heart valve, which is described in further detail with regard to FIGS. 2A, 2B, and 2C below. Preferably, prosthetic coaptation body 200 works together with the native leaflets to both provide a surface for the native leaflets to coapt and to provide a prosthetic valve in a conduit formed by prosthetic coaptation body 200. Unlike prior prosthetic valves that do not use the native leaflets (e.g., because they are cut away or pushed aside by the implant), prosthetic coaptation body 200 may use both the native leaflets and the prosthetic leaflets in the same native heart valve, thereby creating a “double-valve” configuration in the single heart valve. As shown in FIG. 1, support 300 may be structured to suspend and maintain prosthetic coaptation body 200 across the native heart valve once it has been positioned appropriately. As will be understood by one skilled in the art, the illustrated prosthetic coaptation body 200 may be substituted for other prosthetic devices designed to be implanted at a cardiac valve such as a plug/spacer device that coapts with native leaflets to reduce regurgitation such as that shown in U.S. Pat. No. 7,854,762 to Speziali.

As described above, distal, implantable portion 311 of support 300 may be coupled to prosthetic coaptation body 200. Proximal, delivery portion 310 of support 300, may be operatively coupled to actuator 108 and removeably coupled to distal, implantable portion 311 during delivery, such that proximal, delivery portion 310 may be manipulated by actuator 108 to accurately position prosthetic coaptation body 200 across the native valve. Support 300 may have a predefined bend to improve positioning of prosthetic coaptation body 200 across the native valve, as described in further detail below. For example, the bend may be predefined for a specific patient anatomy. Moreover, the predefine bend permits steering of the support from the predefined shape; this may have the effect of reducing stresses and strain on the elongated rail for long-term implant. In addition, heart valve therapeutic device 100 may include one or more radiopaque markers for in-vivo visualization during delivery of prosthetic coaptation body 200.

Referring now to FIGS. 2A, 2B and 2C, exemplary prosthetic coaptation body 200 of heart valve therapeutic device 100 is described. FIG. 2A shows prosthetic coaptation body 200 viewed from the distal end downward, FIG. 2B is a side view of prosthetic coaptation body 200, and FIG. 2C is a cross-sectional view of prosthetic coaptation body 200. Prosthetic coaptation body 200 preferably includes frame 205 having prosthetic leaflets 218 coupled thereto. Prosthetic leaflets 218 may be formed from natural tissue, such as bovine, equine, or porcine pericardial tissue, and/or manmade, synthetic material suitable for implantation such as ePTFE. Prosthetic coaptation body 200 may also contain one or more biocompatible materials, e.g., formed from the natural tissue and/or manmade material, coupled to frame 205 such as skirt 220. Prosthetic leaflets 218 and skirt 220 may be formed from the same material and may be integrally formed from a common piece of material or may be separate. Frame 205 further may include spine connector 221 for coupling with a support connector of support 300 as described in further detail below.

The shape of prosthetic coaptation body 200 is formed by frame 205, which is designed to transition from a contracted, delivery state to an expanded, deployed state and may be formed from shape memory material such as Nitinol. For example, frame 205 may form a conduit that, together with prosthetic leaflets 218 and the biocompatible material covering form a channel to allow blood to travel through prosthetic leaflets 218, when opened during the cardiac cycle, and through prosthetic coaptation body 200.

Skirt 220 may be a thin sheet of biocompatible material surrounding frame 205, extending from proximal ring 206 to distal ring 214 to form the outside surface of the conduit to which the native leaflets coapt when closed during the cardiac cycle. For example, skirt 220 may be sewn to proximal ring 206 and distal ring 214. Skirt 220 may be made of a rigid or compliant material. In some examples, skirt 220 expands and contracts responsive to pressure changes during the cardiac cycle. In this manner, skirt 220 may provide better coaptation with native leaflets. Accordingly, as prosthetic coaptation body 200 sits between the native tricuspid valve leaflets, it fills the regurgitant orifice area caused by right ventricular dilation. The native tricuspid valve leaflets seal against skirt 220 to prevent regurgitation between the native leaflets and prosthetic coaptation body 200 during systole. In addition, prosthetic leaflets 218 integrated within prosthetic coaptation body 200 supports flow during diastole. Prosthetic leaflets 218 may coapt onto the valve frame spine during systole to reduce regurgitation.

Referring now to FIGS. 2D, 2E, and 2F, frame 205 is described. Frame 205 may be made of metal, such as Nitinol or stainless steel. Frame 205 may be made of various components including wires, tubes, or flat strips. In a preferred embodiment, frame 205 is laser cut from one or more tubes of Nitinol. Frame 205 preferably includes spine 201, proximal portion 202 having proximal ring 206 and inner ring 210, and distal portion 204 having distal ring 214. Proximal ring 206 may be coupled to spine 201 via a plurality of proximal tethers 208 and step 203, as described in further detail below with regard to FIG. 2F, and distal ring 214 may be coupled to spine 201 via a plurality of distal tethers 216 and step 207. Alternatively, proximal tethers 208 and distal tethers 216 may be coupled directly to spine 201 without step 203 and step 207, respectively. Spine 201 may be an elongated shaft, e.g., formed from a metal tube such as stainless steel or Nitinol. As shown, spine 201 may extend generally through the middle of prosthetic coaptation body 200 to provide strength and support. In the illustrated example, spine 201 extends through prosthetic coaptation body 200 to the connection with distal ring 214 and proximally past the proximal end of prosthetic coaptation body to permit permanent, secure coupling between spine 201 and support 300. Preferably, proximal tethers 208 and distal tethers 216 are preferably formed of a rigid structure, and are compressible for delivery and may be self-expandable. Inner ring 210 may be positioned distal to and provide additional support to proximal ring 206, and may be coupled to proximal ring 206 via a plurality of slotted prosthetic leaflets anchors 212 having a plurality of suture eyelets to facilitate suturing of prosthetic leaflets 218 (not shown) to frame 205. In a preferred embodiment, inner ring 210 has a diameter less than that of proximal ring 206 and may also have a diameter less than that of distal ring 214.

Proximal ring 206, distal ring 214, and inner ring 210 may have a generally-circular shape, but may be other shapes such as ovals or diamonds. As illustrated, proximal ring 206 may be scallop-shaped such that proximal ring 206 extends circumferentially from prosthetic leaflets anchor 212 proximally and radially outward, then distally and radially inward toward an adjacent prosthetic leaflets anchor 212. In a preferred embodiment, the proximal ends of proximal tethers 208 are coupled to spine 201 via step 203, and extend distally and radially outward such that the distal ends of proximal tethers 208 are coupled to prosthetic leaflets anchors 212. Accordingly, this configuration may optimize prosthetic leaflets shape, e.g., permits prosthetic leaflets anchors 212 to have a longer length with less angulation between spine 201 and proximal tethers 208, thereby reducing material strains and stresses during locking of the distal, implantable portion of the support. Advantageously, the frame and ring structures are expected to maximize coaptation length while minimizing valve length and/or to create a coronary sinus-like region around the leaflet area (e.g., leaflet scallops) to maximize washout.

Inner ring 210 may extend circumferentially from prosthetic leaflets anchor 212 distally and radially outward, then proximally and radially inward toward an adjacent prosthetic leaflets anchor 212. Accordingly, inner ring 210 provides additional rigidity to proximal ring 206 and prosthetic leaflets anchors 212, which permits a reduction of overall size of frame 205, thereby reducing stress during collapse of frame 205, and improving durability of prosthetic coaptation body 200. As illustrated, distal ring 214 preferably has a sinusoidal wave shape around its circumference, such that distal tethers 216 are preferably coupled to distal ring 214 at a valley of the sinusoid. Distal ring 214 may have other oscillating shapes, which may be different in form to proximal ring 206. Thus, the proximal ends of distal tethers 216 are coupled to spine 201 via step 207, and extend distally and radially outward such that the distal ends of distal tethers 216 are coupled to the valley of distal ring 214.

As shown in FIG. 2F, the proximal ends of proximal tethers 208 may be coupled to spine 201 via step 203. Step 203 may be constructed from Nitinol and may be laser welded into place on spine 201, or alternatively, via molding or adhesive. Step 203 may have a wall thickness that is smaller, larger, or equal to the wall thickness of the cut tube from which frame 205 is formed. In addition, step 203 may include relief cuts to permit step 203 to be opened out onto spine 201 for optimal clearances for welding. Step 207 may be constructed similar to step 203.

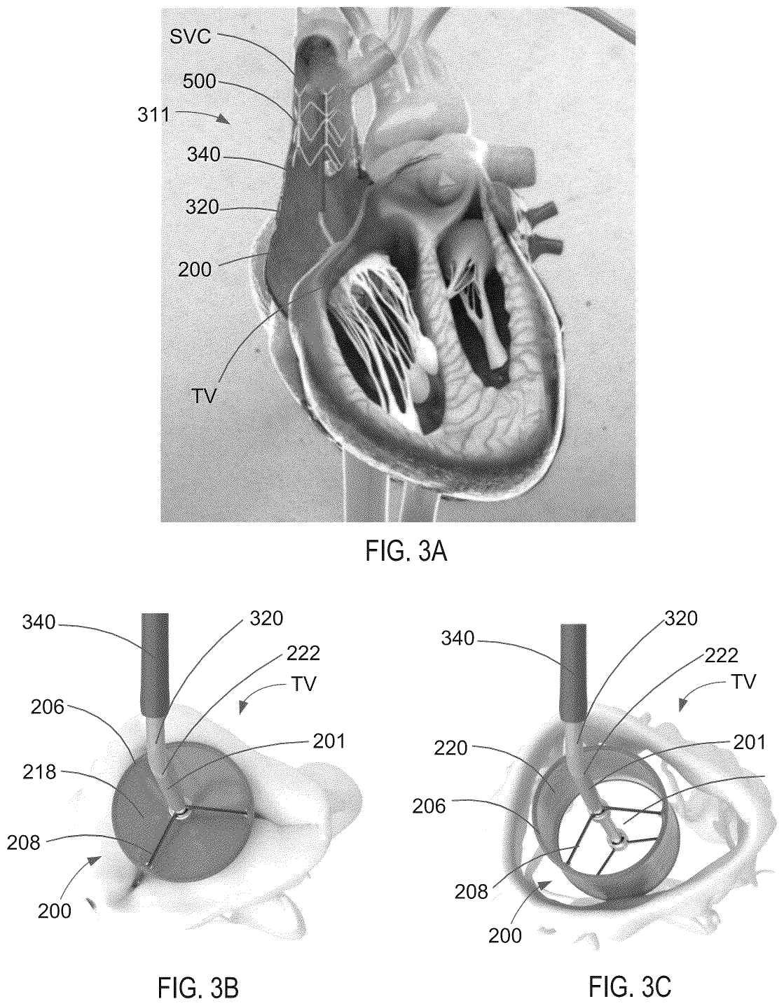

FIGS. 3A to 3C illustrate prosthetic coaptation body 200 implanted at a native heart valve. As shown in FIG. 3A, prosthetic coaptation body 200 is suspended within the native heart valve via distal, implantable portion 311 of support 300 and anchor 500. For example, anchor 500 may be implanted within SVC such that prosthetic coaptation body 200 is suspended within tricuspid valve TV via body support catheter 320, as described in further detail below. FIG. 3B shows prosthetic coaptation body 200 suspended within the tricuspid valve TV during systole whereby prosthetic leaflets 218 are in a closed configuration and the native valve leaflets are sealed against the outer surface of prosthetic coaptation body 200 to prevent regurgitation between the native leaflets and prosthetic coaptation body 200. FIG. 3C shows prosthetic coaptation body 200 suspended within the tricuspid valve TV during diastole whereby prosthetic leaflets 218 and the native valve leaflets are in an open configuration, thereby permitting flow therethrough. Advantageously, the prosthetic device sits across the native valve and contacts the native leaflets when they seal during systole, but the prosthetic device need not be in contact with the annulus. The anchoring system sits above the prosthetic device in the atrium and may extend up to the stent in the SVC or, alternatively, the IVC.

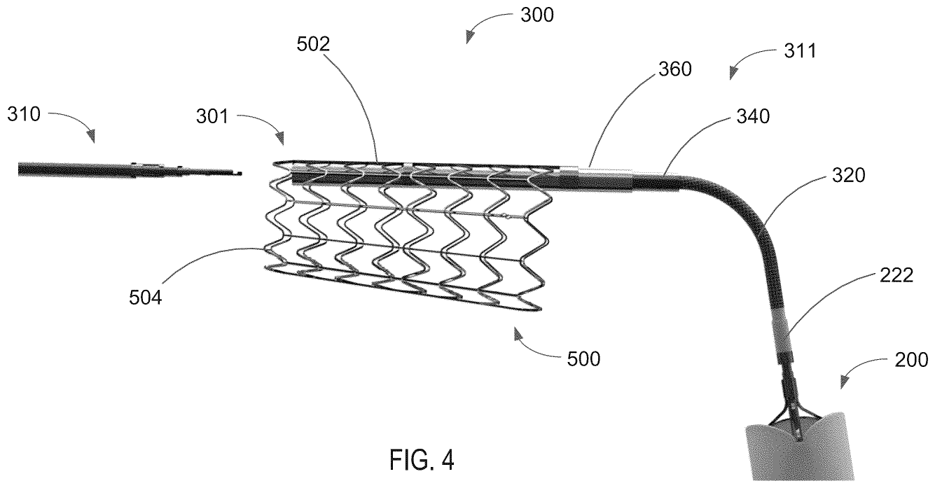

Referring now to FIG. 4, support 300 for delivering and implanting the prosthetic coaptation body is described. Support 300 includes an elongated shaft including proximal, delivery portion 310 and distal, implantable portion 311, and may be steered and manipulated via actuator 108 (see FIG. 1). Proximal, delivery portion 310 may be removeably coupled to distal, implantable portion 311 at detachment area 301 during delivery of prosthetic coaptation body 200, and proximal, delivery portion 310 may be decoupled from distal, implantable portion 311 to thereby implant distal, implantable portion 311 and prosthetic coaptation body 200 in the desired location within the patient. Accordingly, proximal, delivery portion 310 may be removed from the patient, while distal, implantable portion 311 and prosthetic coaptation body 200 remain fully implanted. As shown in FIG. 4, distal, implantable portion 311 of support 300 includes valve connection area 222 for coupling with prosthetic coaptation body 200 (e.g., via spine connector 221), anchor 500 including stent 504 coupled to anchor tube 360 via anchor support 502, and a plurality of catheters including body support catheter 320 and shaping catheter 340 for maneuvering and implanting prosthetic coaptation body 200, as described in further detail below.

As described above, the elongated rail may extend from actuator 108 to prosthetic coaptation body 200, and may have a pre-formed bend area to facilitate delivery of prosthetic coaptation body 200 to the native heart valve. In addition, the pre-formed bend may reduce the stress required to position prosthetic coaptation body 200 during delivery. For example, the elongated rail may be an elongated shaft made of metal (e.g., Nitinol) that is preformed to a predetermined angle (e.g., 50-150 degree bend, 100 degree bend). The body support catheter may be coaxial to the preformed rail and the shaping catheter and may be attached to the prosthetic device to facilitate telescoping of the prosthetic device beyond the bend. The shaping catheter may be coaxial to the preformed rail and the body support catheter. In some embodiments, the distal end of the shaping catheter may have a collar that is used to bend and straighten the preformed rail based on the relative axial position between the two responsive to actuation at the handle. For example, as the shaping catheter is advanced distally over the preformed bend of the elongated rail, the elongated rail straightens, and as the shaping catheter is retracted proximally relative to the elongated rail, the elongated rail returns to its natural state with the preformed bend. As will be understood by a person having ordinary skill in the art, the rail may be moved while the shaping catheter remains stationary within the patient to bend and straighten the preformed rail.

The prosthetic device may be secured on the distal end of the anchor system using the implantable support catheter connected to a Nitinol Stent with a disconnectable proximal section to support delivery. The support catheter may be used to deliver and adjust and finally stabilize the position the prosthetic device across the native cardiac valve. The anchor system may be used to deploy, position, and support the prosthetic device, attached to the distal end. Once the prosthetic device has been positioned, a self-expanding Nitinol stent may be deployed in the tissue (e.g., SVC or IVC) which may be attached to the support catheter. The position of the prosthetic device can be further adjusted after the stent is deployed. The stent may be supported by the anchor tube component of the support catheter. The support catheter may have a steerable distal portion that is controlled by the handle to ensure optimal positioning. This can avoid hooks, screws or clamps in the thin, frail structures of the dilated right heart and allows the system to accommodate cardiac and respiratory motion. After deployment and final positioning of the prosthetic device, the position is locked and the delivery section of the anchor system, proximal to the stent, is disconnected and removed.

Advantageously, the anchor system can allow for deflection from a straight configuration through 100 degrees of angulation so that the prosthetic device can be positioned coaxial to the tricuspid annulus; telescoping of the prosthetic device down, towards the apex of the ventricle, into the tricuspid annulus so that it is positioned properly between the tricuspid valve leaflets; extension and rotation of the position of the bend relative to the stent so that the prosthetic device can cross the tricuspid valve perpendicular to it, and so the clinician may freely position the stent to a preferred location; stabilizing of the prosthetic device in position by anchoring against the tissue (e.g., wall of a blood vessel such as the SVC or IVC); fixing the selected position, angulation and telescoping, of the prosthetic device. The positioning of the prosthetic device is helped by the native valve leaflets which naturally direct and center it within the central gap of the leaflets. The distal portion of the anchor system has sufficient stiffness to maintain the prosthetic device in position during the cardiac cycle, as well as sufficient flexibility to permit the prosthetic device to “self-center” within the native valve during systole. This distal portion of the anchor system may be connected to support the prosthetic device and stabilized in the SVC by the stent anchor. The sterilization process for the anchor system and its accessories may be Ethylene Oxide (ETO) or radiation sterilized. This sterilization process is standard for catheter systems.

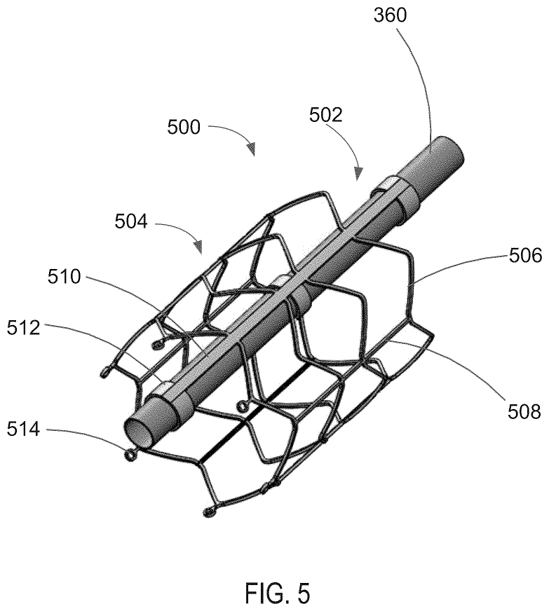

Referring now to FIG. 5, an exemplary anchor is described. As shown in FIG. 5, anchor 500 includes stent 504 coupled to anchor tube 360 via anchor support 502. Anchor tube 360 has a lumen sized and shaped to receive shaping catheter 340 (not shown) therethrough. Anchor tube 360 may be coupled to one side of stent 504 to stabilize distal, implantable portion 311 of support 300 and bias it to one side of the blood vessel. For example, stent 504 may include longitudinal stent spine 510. Stent spine 510 may be formed with stent 504 as a single component, or may be a separate component affixed to stent 504. Stent spine 510 may be coupled to one or more anchor tube cuffs 512 for clamping stent 504 to anchor tube 360. For example, anchor tube cuffs 512 may have a lumen sized and shaped to receive anchor tube 360 therethrough. Anchor tube cuffs 512 may be coupled to anchor tube 360 such that relative movement between anchor tube cuffs 512 and anchor tube 360 is prevented. Although only three anchor tube cuffs 512 are illustrated in FIG. 5, a person having ordinary skill in the art would understand that anchor tube cuffs 512 may include less than three cuffs, e.g., one or two cuffs, or more than three cuffs, e.g., four, five, six cuffs or more as necessary.

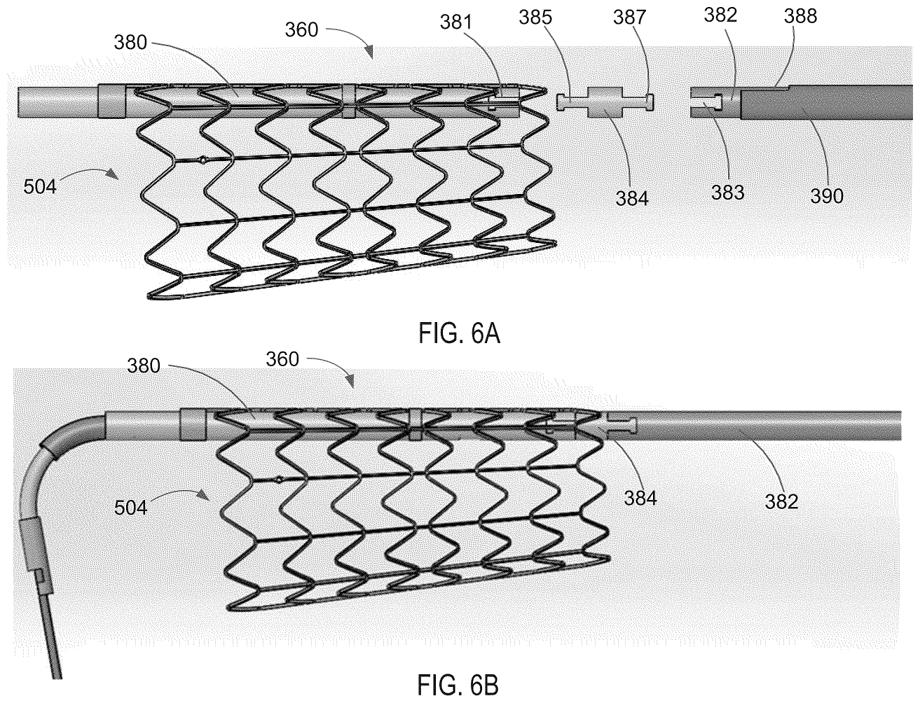

Referring now to FIGS. 6A and 6B, an exemplary anchor tube is described. FIG. 6A illustrates the components of anchor tube 360 separated for clarity. As shown in FIG. 6A, support 300 may include anchor tube 360 having anchor tube distal portion 380 at distal, implantable portion 311 of support 300, and anchor tube proximal portion 382 at proximal, delivery portion 310 of support 300. Anchor tube 360 may extend from actuator 108 toward prosthetic coaptation body 200 where it may be coupled to stent 504. Anchor tube proximal portion 382 may be attached to anchor tube distal portion 380 via anchor tube connection 384 during delivery. For example, anchor tube distal portion 380 may have distal anchor tube connection portion 381 having a first geometry, and anchor tube connection 384 may have distal anchor tube interlinking portion 385 having a second geometry corresponding with the first geometry of distal anchor tube connection portion 381 such that, in the delivery configuration, distal anchor tube connection portion 381 engages with distal anchor tube interlinking portion 385.

In addition, anchor tube proximal portion 382 may have proximal anchor tube connection portion 383 having a third geometry, and anchor tube connection 384 may have proximal anchor tube interlinking portion 387 having a fourth geometry corresponding with the third geometry of proximal anchor tube connection portion 383 such that, in the delivery configuration, proximal anchor tube connection portion 383 engages with proximal anchor tube interlinking portion 387. As shown in FIG. 6A, anchor tube 360 further may include anchor tube sleeve 390, which may be slidably disposed over at least anchor tube proximal portion 382 and anchor tube connection 384. FIG. 6B illustrates the components of anchor tube 360 in an engaged delivery position, without anchor tube sleeve 390 for clarity.

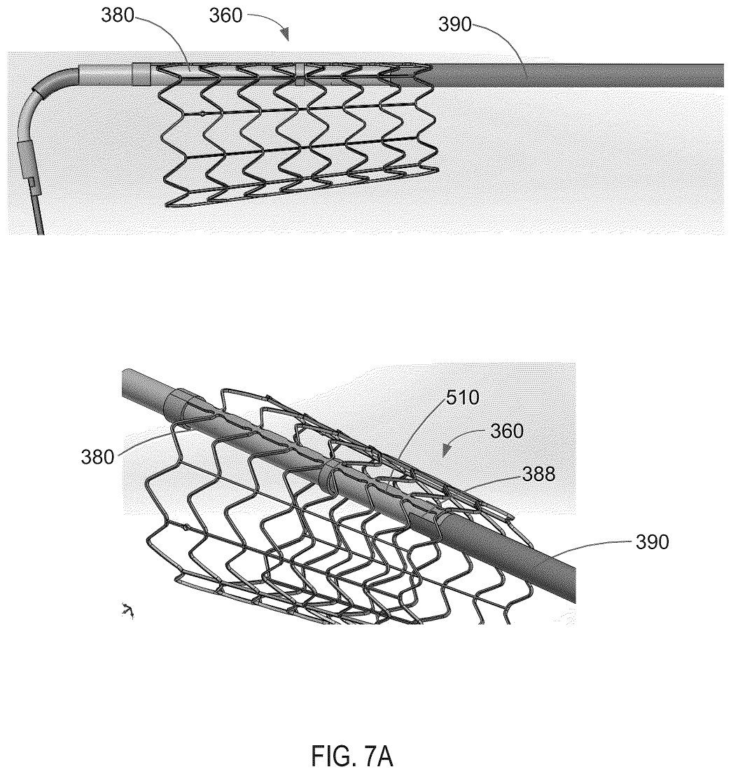

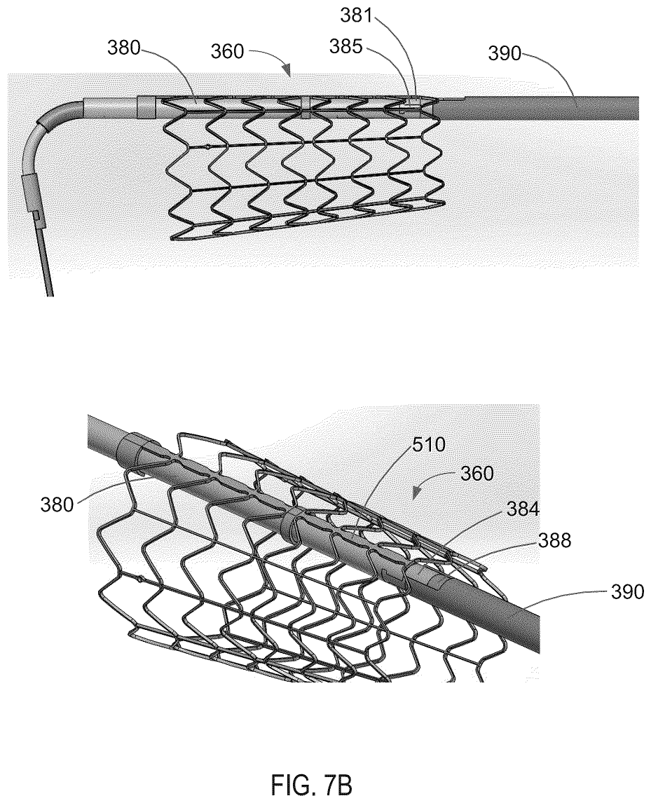

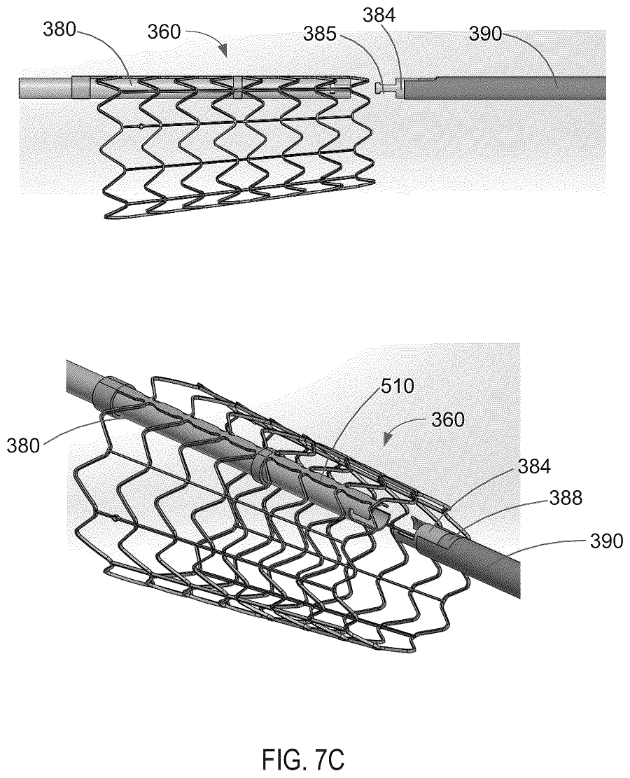

Referring now to FIGS. 7A to 7C, an exemplary method for detaching the proximal components of anchor tube 360 from anchor tube distal portion 380 such that anchor tube distal portion 380 may be implanted within the patient is described. As shown in FIG. 7A, in the delivery configuration, anchor tube sleeve 390 is disposed over anchor tube proximal portion 382 and anchor tube connection 384 while anchor tube connection 384 is engaged with anchor tube distal portion 380. As shown in FIGS. 7A to 7C, the distal end of anchor tube sleeve 390 may include opening 388, such that when anchor tube sleeve 390 is disposed over anchor tube connection 384 in the delivery configuration, anchor tube sleeve 390 avoids collision with the stent spine of the stent of anchor 500. Next, as shown in FIG. 7B, anchor tube sleeve 390 may be retracted proximally responsive to actuation of actuator 108, e.g., by actuating an interface on actuator 108, to expose distal anchor tube interlinking portion 385 of anchor tube connection 384. Distal anchor tube interlinking portion 385 may self-expand from a collapsed delivery state within anchor tube sleeve 390 to an expanded state upon exposure from anchor tube sleeve 390, such that distal anchor tube interlinking portion 385 disengages with anchor tube distal portion 380 at distal anchor tube connection portion 381. As shown in FIG. 7C, anchor tube proximal portion 382, anchor tube connection 384, and anchor tube sleeve 390 may be removed from the patient while anchor tube distal portion 380 remains implanted within the patient. Preferably, retraction of anchor tube sleeve 390 is limited such that anchor tube sleeve 390 remains disposed over proximal anchor tube interlinking portion 387 and anchor tube proximal portion 382. In some embodiments, proximal anchor tube interlinking portion 387 and anchor tube proximal portion 382 may be permanently linked.

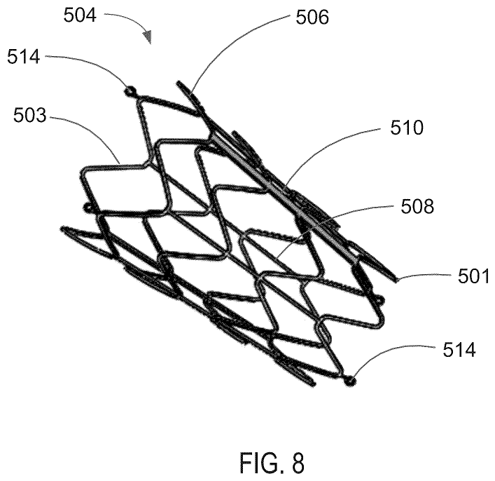

Referring now to FIG. 8, an exemplary stent is described. As shown in FIG. 8, stent 504 may be formed by a plurality of sinusoidal or zig-zag or otherwise oscillating circumferential pattern of struts 506, interconnected via a plurality of longitudinal struts 508. One of the plurality of longitudinal struts 508 may be stent spine 510, or alternatively, stent spine 510 may be affixed to one of the plurality of longitudinal struts 508. Although only four circumferential struts 506 are illustrated in FIG. 8, a person having ordinary skill in the art would understand that stent 504 may include less than four circumferential struts, e.g., two or three circumferential struts, or more than four circumferential struts, e.g., five, six, seven, eight circumferential struts or more as necessary. In accordance with one aspect, the pattern of struts 506 and longitudinal struts 508 are formed such that the length of stent 504 may be the same in its collapsed, delivery state within the delivery sheath, as in its expanded, deployed state.

Stent 504 may be self-expandable such that stent 504 transitions from a collapsed, delivery state within a delivery sheath, to an expanded, deployed state within the target blood vessel for anchoring support 300. Stent 504 may have a variable stiffness around its circumference and along its length. For example, the width of the frame forming circumferential struts 506, the longitudinal length of a strut of the plurality of circumferential struts 506, and/or the radius of curvature of the plurality of circumferential struts 506 may be varied to achieve the desired stiffness of stent 504. In addition, stent 504 may have a plurality of loops for radiopaque markers, e.g., gold markers 514, to assist in visualization during delivery of prosthetic coaptation body 200. As shown in FIG. 8, proximal end 501 of stent 504 may have a smaller diameter than distal end 503 of stent 504. This tapered configuration may improve conformability with a tapered vessel, reduce risk of trauma to the tapered vessel, and provide more even oversizing over the length of the stent in the tapered vessel. This tapered configuration further may improve anchoring within a patient's blood vessel. Moreover, stent 504 may have a plurality of barbs extending from its outer surface, and/or small local flares and/or flared cells as described in further detail below, to improve migration resistance. In a further embodiment, the stent may have a variable diameter along its length to improve anchoring.

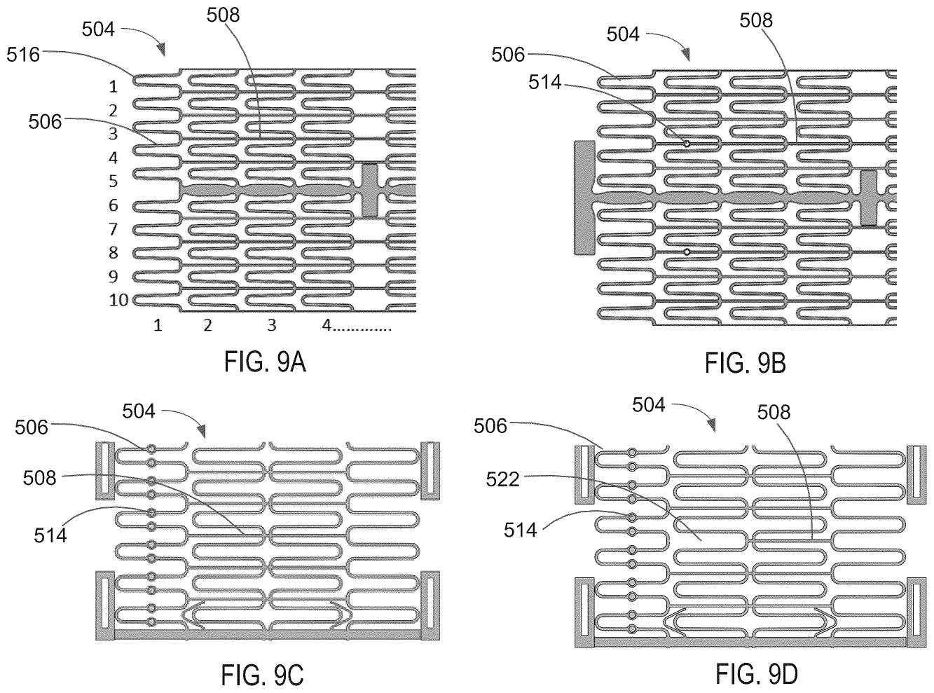

FIGS. 9A to 9D illustrate various configurations of the frame of the stent of the anchor. As shown in FIG. 9A, circumferential struts 506 of stent 504 may include N numbers of sinusoids 516 along its circumferential length, and stent 504 may include N number of circumferential struts 506 along its longitudinal length. As shown in FIG. 9B, markers 514 may be positioned along plurality of longitudinal struts 508. Alternatively, as shown in FIG. 9C, markers 514 may be positioned along plurality of circumferential struts 506. As shown in FIG. 9D, longitudinal struts 508 may not extend across every circumferential struts 506, thereby creating space 522 without a longitudinal strut extending thereacross, which may improve distribution of strain on stent 504 and conformability to the vessel. In some embodiments, stent 504 may not have any longitudinal struts.

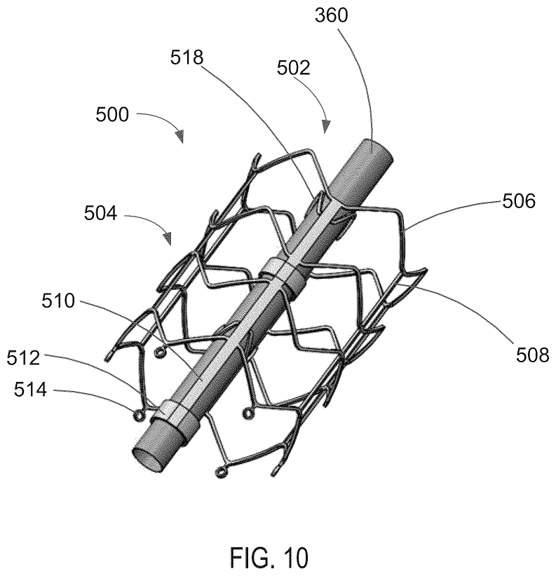

FIG. 10 illustrates anchor 500 having an alternative coupling mechanism between stent spine 510 and anchor tube 360. For example, anchor 500 further may include a plurality of V-shaped cuffs 518 for clamping stent spine 510 to anchor tube 360. As shown in FIG. 10, anchor 500 may include V-shaped cuffs 518 in addition to anchor tube cuffs 512. Alternatively, anchor 500 may only include V-shaped cuffs 518 and no anchor tube cuffs 512. Moreover, any number of V-shaped cuffs 518 and/or anchor tube cuffs 512 may be used to stabilize stent 504 relative to anchor tube 360.

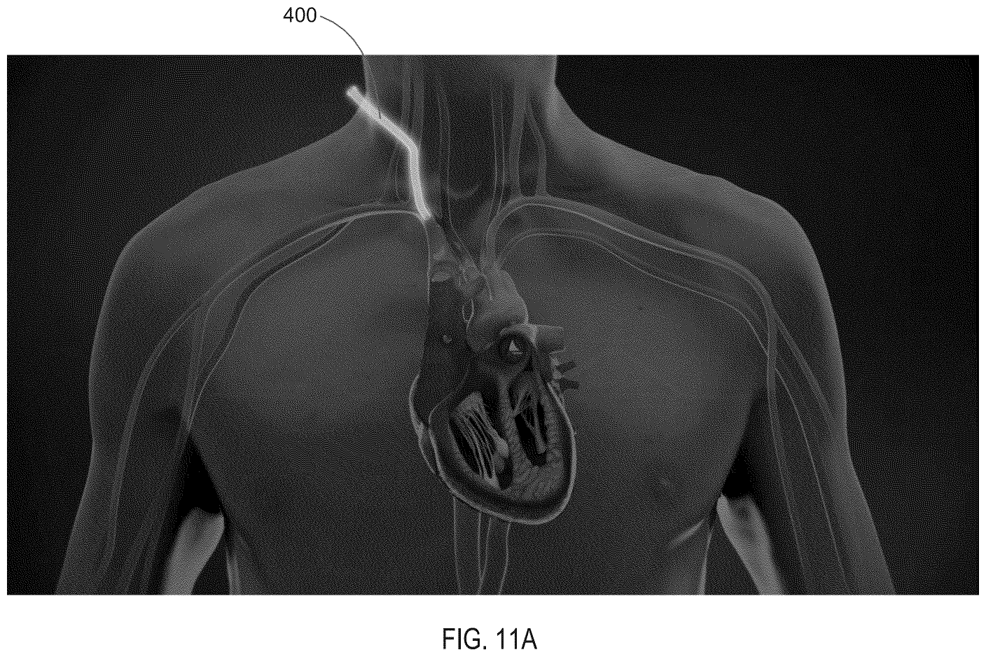

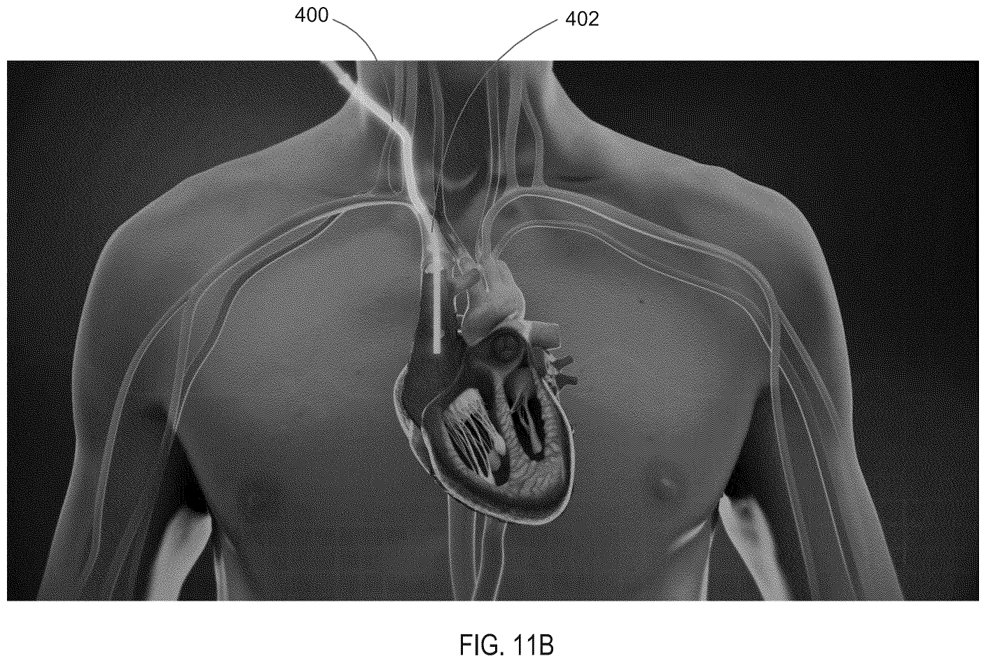

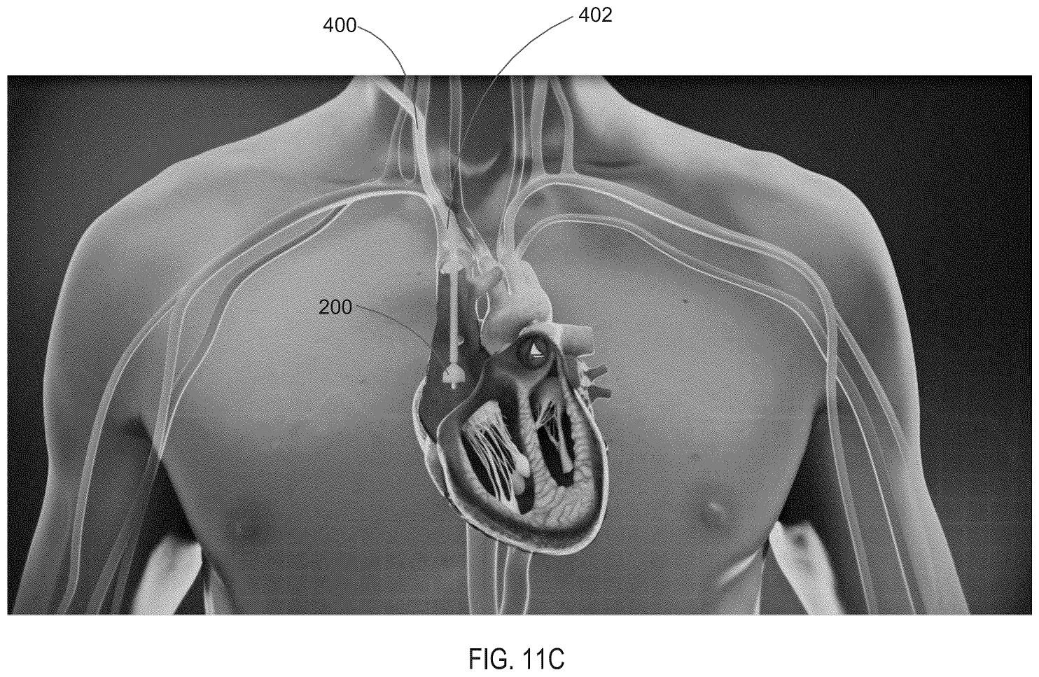

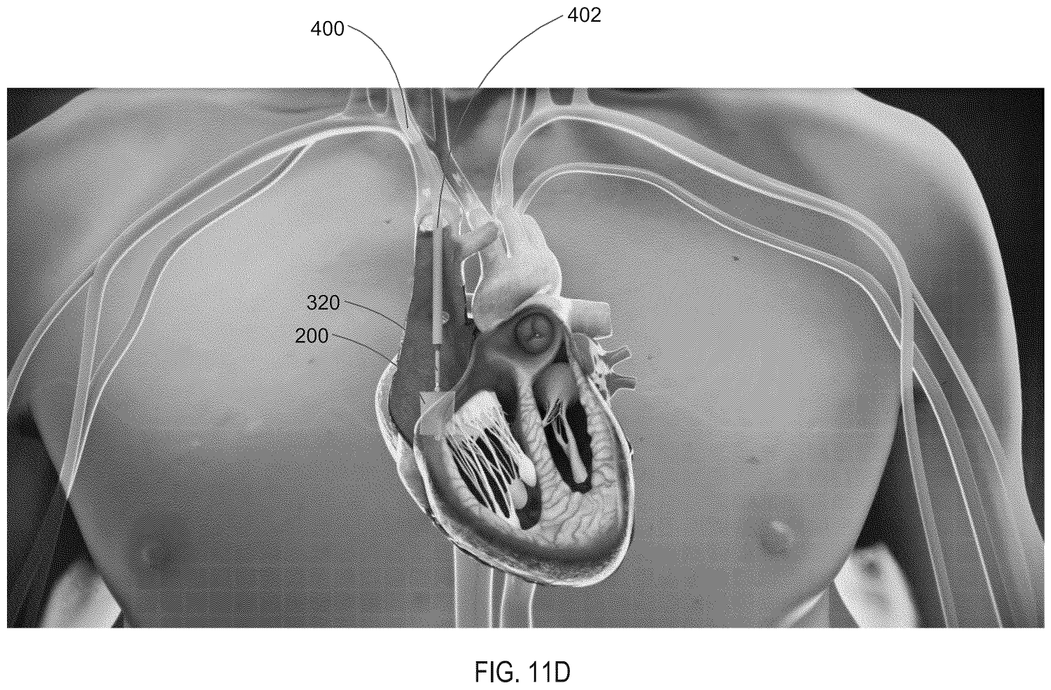

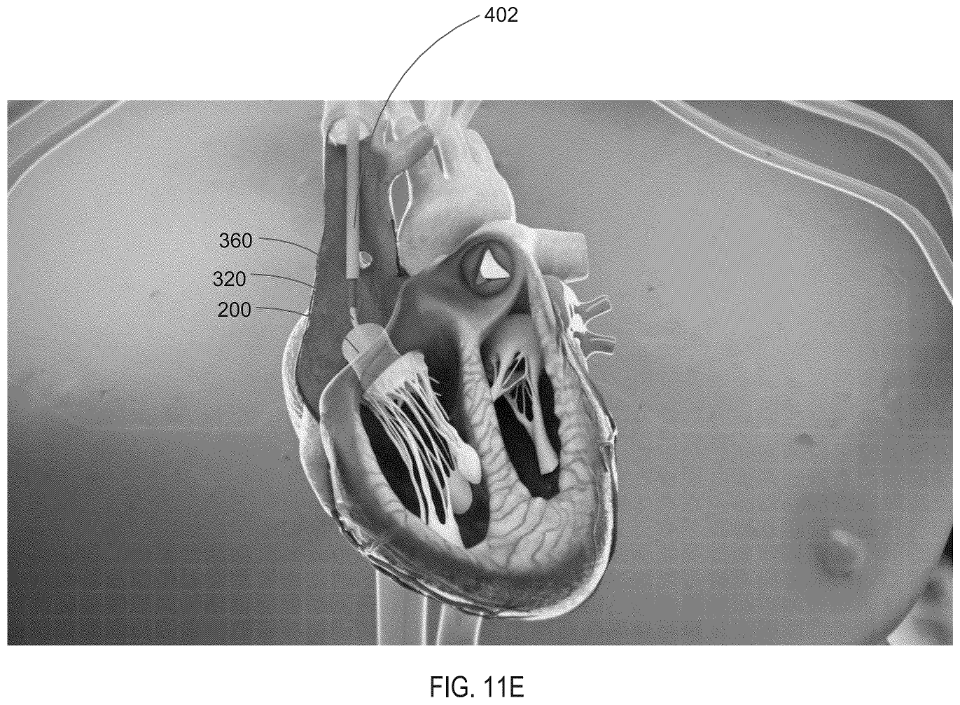

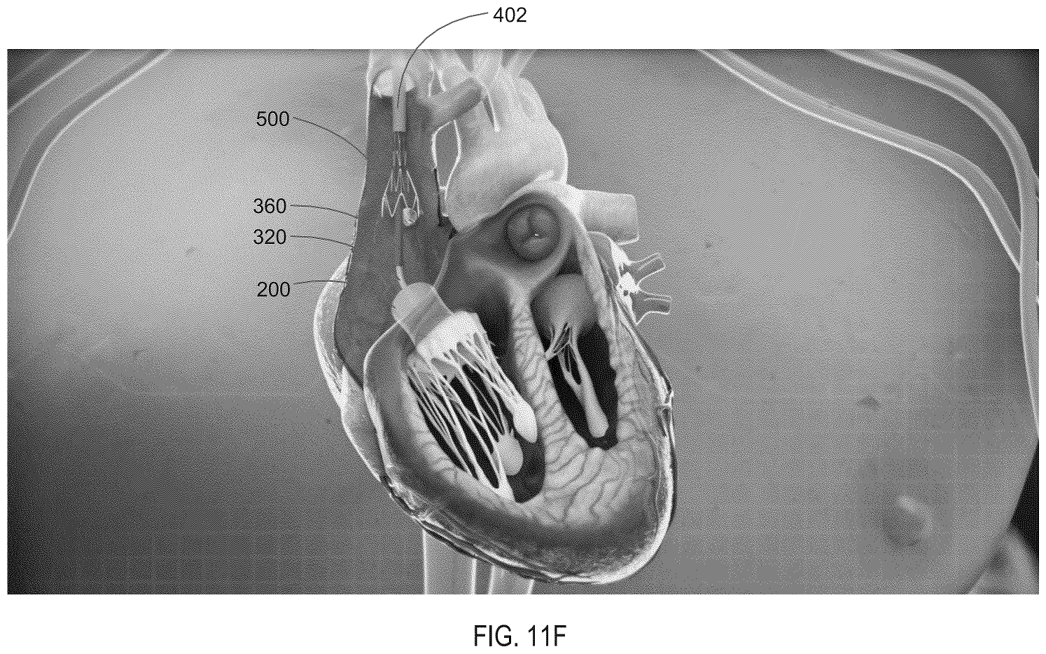

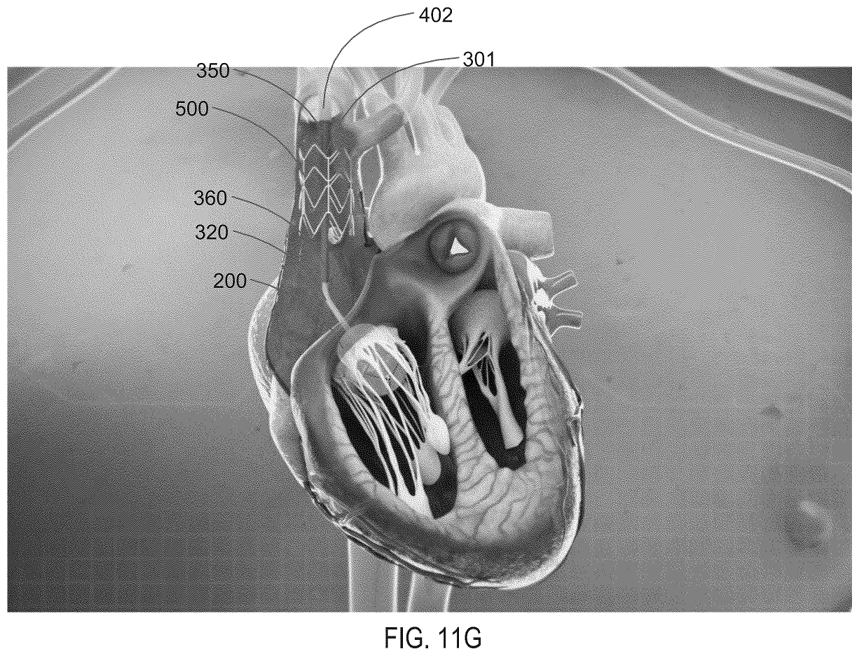

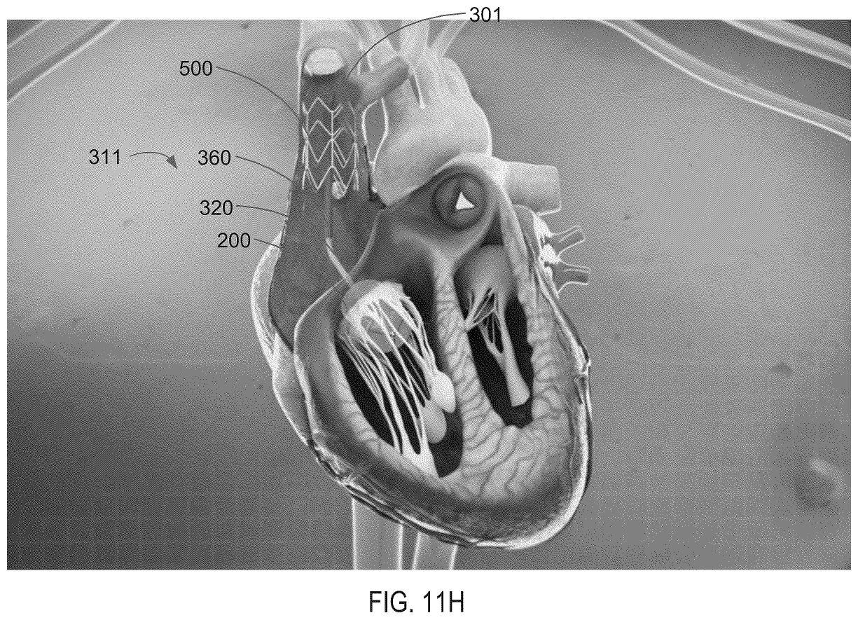

Referring now to FIGS. 11A to 11H, an exemplary method of inserting and positioning prosthetic coaptation body 200 across a native valve is shown. Prior to the implantation procedure, the patient may undergo a cardiac gated-CT to define the right heart and SVC anatomy. For the procedure, the patient may be fully anesthetized and may undergo right atrial and ventricular angiography. A sheath (e.g., 26 French sheath) may be inserted into the right internal jugular vein and a femoral venous (e.g., 9 Fr) line may be inserted for delivery of an intracardiac echo probe. All venous access may be obtained by ultrasound guidance. As shown in the figures described below, the prosthetic device may be deployed out of the delivery sheath and into the right atrium. The anchor system may then be manipulated under x-ray guidance to increase the bend angle and advance the prosthetic device until it crosses the tricuspid annulus. Once the initial device position is achieved with the prosthetic device across the tricuspid valve, a sheath may be retracted further to deploy the stent in the SVC and then positioning may be further adjusted to determine the final, optimal position. Correct device positioning may be confirmed by fluoroscopy and echocardiography. Clinical, hemodynamic, and echocardiographic outcomes may be assessed serially during the procedure to achieve optimum position. Echocardiography may be performed at baseline and after device placement to assess device function and tricuspid regurgitation. Once the optimal position is achieved, the locks in the handle may be released to lock and detach the system (e.g., a plurality of locks such as two locks and a plurality of disconnected elements such as four disconnected elements), and the handle and sheath can then be removed.

In FIG. 11A, sheath introducer 400 is inserted percutaneously into a blood vessel, e.g., through the jugular vein near a patient's neck or via the femoral artery. Sheath introducer 400 provides an opening for a clinician to percutaneously insert prosthetic coaptation body 400 and support 300 into the blood vessel. In FIG. 11B, delivery sheath 402 is inserted through sheath introducer 400 and advanced distally into the area in which prosthetic coaptation body 200 will be deployed. In the case of a damaged tricuspid valve, for example, delivery sheath 402 will extend through the inferior or superior vena cava into the right atrium such that the distal end of delivery sheath 402 is in the right atrium. In some cases, a guide wire may be introduced prior to inserting delivery sheath 402 to guide delivery sheath 402 through the blood vessel. Delivery sheath 402 preferably contains prosthetic coaptation body 200 in its compressed delivery state and support 300. Preferably, anchor 500 and anchor tube 360 may also be contained in delivery sheath 402, where anchor 500 is in a compressed delivery state.

As illustrated in FIG. 11C, prosthetic coaptation body 200 is moved distally, e.g., using actuator 108 and/or interfaces 110, 112, 114, 116, 118, 120 out the distal end of delivery sheath 402, exposing prosthetic coaptation body 200, which expands to an expanded deployed state. For example, a clinician may move actuator 108 distally while holding delivery sheath 402 in place such that the prosthetic device moves out the distal end of the sheath and self-expands upon deployment. Support 300 may alternatively be held in place while delivery sheath 402 is withdrawn to expose prosthetic coaptation body 200. FIG. 11D shows prosthetic coaptation body 200 in its fully expanded state. As illustrated in FIG. 11E, body support catheter 320 may be steered to orient prosthetic coaptation body 200 in its deployed orientation using actuator 108 and/or one or more interfaces 110, 112, 114, 116, 118, 120.

Anchor tube 360 is partially exposed, either by being pushed through delivery sheath 402 or by withdrawing delivery sheath 402 while holding in place anchor tube 360. As illustrated in FIG. 11F, delivery sheath 402 is further withdrawn, exposing anchor 500, which expands upon exposure from delivery sheath 402. As illustrated in FIG. 11G, anchor 500 is fully exposed and expanded, and engages with the walls of a blood vessel, holding itself in position. In the case of a damaged tricuspid valve, as shown, anchor 500 may engage the walls of the superior vena cava. Prosthetic coaptation body 200 may then be moved into its final deployed position using actuator 108 and/or interfaces 110, 112, 114, 116, 118, 120. For example, the interface operatively coupled to the shaping catheter may be actuated to adjust extension of the shaping catheter relative to the anchor tube to extend prosthetic coaptation body 200 to the desired distance within the native heart valve.

In addition, the interface operatively coupled to the elongated rail may be actuated to adjust the angle of the elongated rail relative to the shaping catheter such that prosthetic coaptation body 200 is a positioned at the desired angle relative to anchor 500. Moreover, the interface operatively coupled to body support catheter 320 may be actuated to telescope the body support catheter relative to the elongated rail to position prosthetic coaptation body 200 in the desired position within the native valve. Additionally, the catheters of support 300 may be rotated, e.g., by rotating actuator 108, relative to anchor 500. Once prosthetic coaptation body 200 is properly positioned, the locking and disengagement process described above may be implemented to lock the distal components of support 300 together, and disengage the proximal components of support 300 from the distal components at the detachment area so that the proximal components may be removed from the patient, as described in U.S. Pat. No. 11,219,525.

As will be understood by a person having ordinary skill in the art, when detachment of the proximal components from the distal components of the support does not require self-expanding connections, the detachment of the elongated rail, body support catheter, shaping catheter, and anchor tube may be performed independently and in any order. Because anchor tube 360 is coupled to anchor 500, prosthetic coaptation body 200 will remain in place suspended across the native valve. As illustrated in FIG. 11H, distal, implantable portion 311 remains implanted while proximal, delivery portion 310, delivery sheath 402, and sheath introducer 400 are withdrawn, e.g., by pulling actuator 108 proximally.

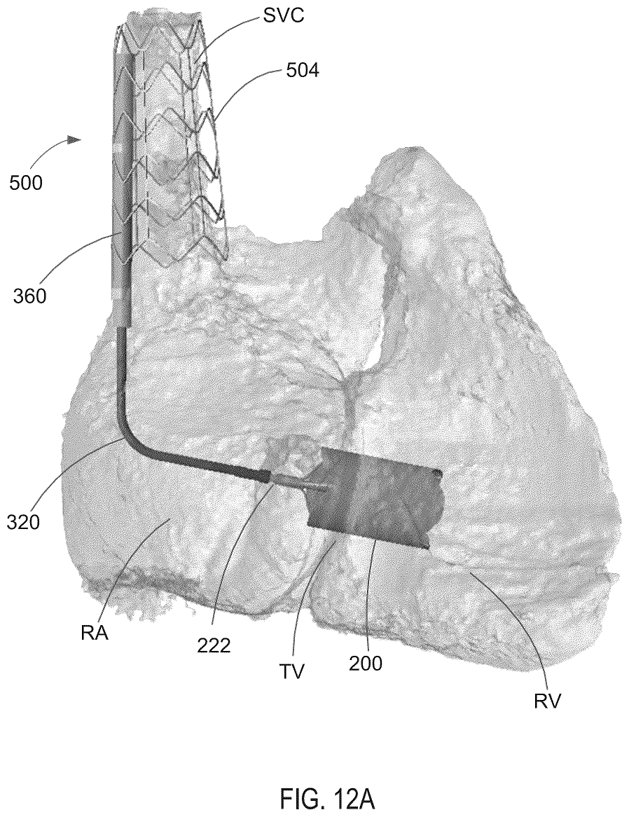

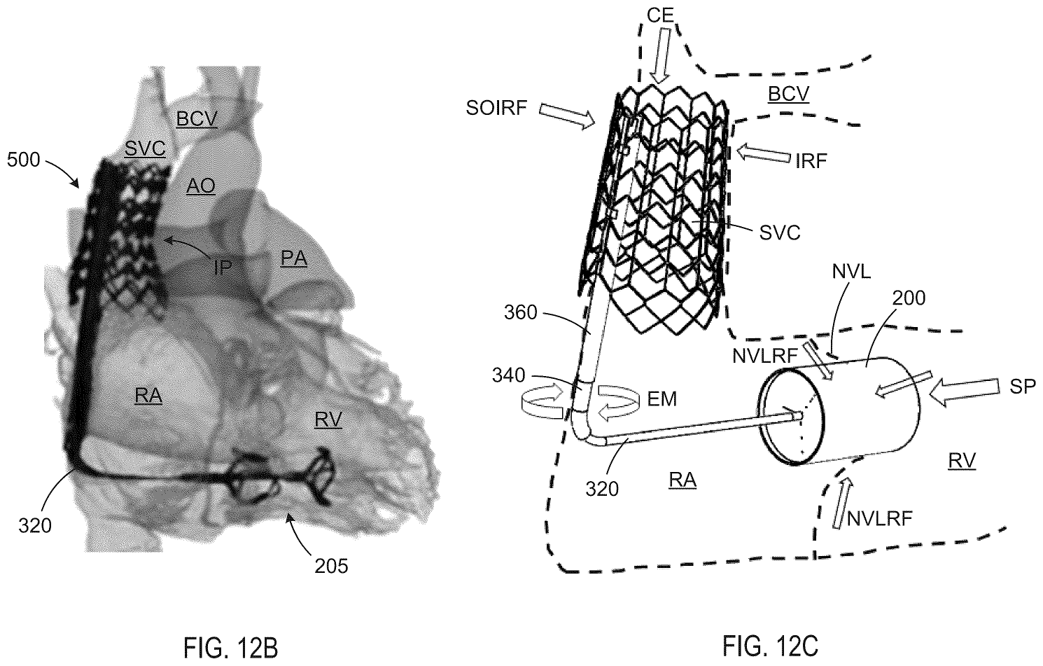

FIG. 12A illustrates the prosthetic coaptation body 200 in an implanted, deployed state for treating cardiac valve regurgitation. As shown in FIG. 12A, anchor 500 is implanted in the superior vena cava SVC while the support extends into the right atrium RA, bends a predefined angle (e.g., about 100 degrees) toward right ventricle RV, such that prosthetic coaptation body 200 is positioned across the tricuspid valve TV. FIG. 12B illustrates anatomical structures adjacent to anchor 500 implanted within superior vena cava SVC including, for example, brachiocephalic vein BCV, aorta AO, and pulmonary artery PA. As shown in FIG. 12B, the variation of tortuosity of superior vena cava SVC and relative positions of aorta AO and pulmonary artery PA may generate local inflection point IP in the stent of anchor 500. By oversizing the stent relative to superior vena cava SVC, inflection point IP may provide counter interacting distal forces to resist migration forces and maintain anchor 500 at the target implant location within superior vena cava SVC.

As shown in FIG. 12C, due to the tapered shape of superior vena cava SVC with a larger opening towards the inferior (e.g., distal) direction which results in significant inferior reaction forces IRF due to significant compression on the cranial (e.g., proximal) end of the stent relative to the inferior end of the stent as well as inferior resultant forces SOIRF generated by oversizing of the stent relative to superior vena cava SVC, the reaction forces NVLRF of the native valve leaflets NVL during each cardiac cycle onto prosthetic coaptation body 200, the pressure SP applied onto prosthetic coaptation body 200 during systole, a crawling effect CE may be applied on anchor 500, such that if the inflection point on the stent of anchor 500 is not pronounced enough, the crawling effect CE may eventually lead to migration of anchor 500, e.g., towards right atrium RA. In addition, the tapered geometry of the stent, which is designed to be less traumatic to the tapered superior vena cava SVC may further contribute to the generation of distally acting reaction forces applied to the stent depending on the variation in the level of compression on the stent due to its oversizing. Moreover, as support 300 transfers the motion of prosthetic coaptation body 200 (which is displaced laterally and axially during coaptation with native valve leaflets NVL and pressure variation during systole and diastole) to anchor 500, this transfer of motion may result in elliptical motion EM onto the inferior end of stent tube 360. In combination with the geometry of the stent and the forces applied thereto as discussed above, the rotational forces applied to support 300 may result in a worming effect which may eventually gradually translate into inferior migration of the stent, e.g., in a direction towards the heart.

To reduce the risk of migration of the stent within the SVC, e.g., in a distal direction towards the heart, the cranial end of the stent may be placed close to, e.g., above, or aligned with the confluence of brachiocephalic vein BCV from superior vena cava SVC, without extending completely across the confluence, as shown in FIG. 12C. As this approach may reduce the treatable population, a larger landing window for the stent within the SVC is preferable. In accordance with some aspects, the addition of antimigration features may enlarge the landing window and remove the need of the specifics with regard to the inflection point generated by the aorta onto the stent, as described in further detail below.

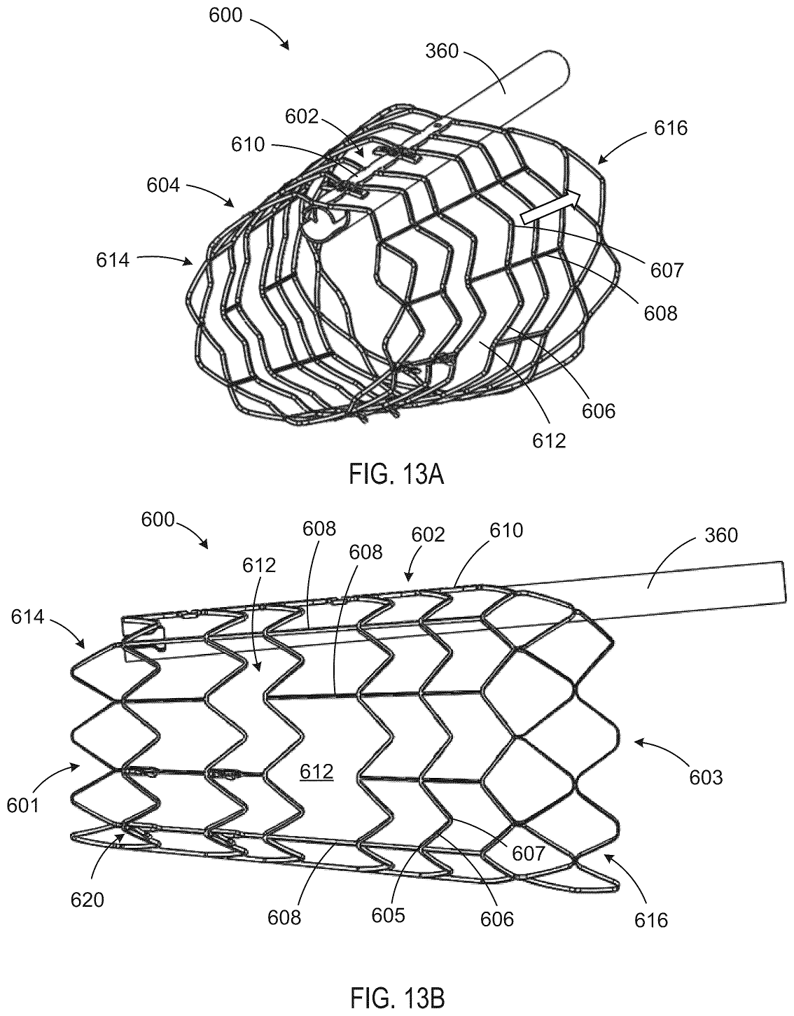

Referring now to FIGS. 13A to 13D, an alternative exemplary anchor for maintaining the prosthetic device at the native cardiac valve is provided. Like anchor 500, anchor 600 may be formed of a stent structure that is transitionable between a contracted, delivery state and expandable to an expanded, deployed state where anchor 600 contacts the inner wall of a blood vessel (e.g., the SVC or IVC) to anchor the prosthetic device at the native cardiac valve. Accordingly, in the contracted, delivery state, anchor 600 may be disposed within a delivery sheath for delivery via, e.g., an intrajugular approach. As shown in FIGS. 13A and 13B, anchor 600 includes stent 604 coupled to anchor tube 360 via anchor support 602, e.g., via longitudinal stent spine 610, which may be formed with stent 604 as a single component, or may be a separate component affixed to stent 604. Stent 604 may be rigidly connected to anchor tube 360 (e.g., via welded or alternative joining processes), and may be formed of shape memory material, e.g., Nitinol.

As shown in FIG. 13B, stent 604 may have a tapered profile with a larger opening at its distal end 603 relative to its opening at its proximal/cranial end 601, which may permit better conformability with the overall tapered profile of the SVC. Moreover, the tapered profile of stent 604 further allows for easier recapture of stent 604, e.g., when stent 604 is not fully exposed from the delivery sheath. Stent 604 may be formed by a plurality of circumferentially-extending struts 606, selectively interconnected via a plurality of longitudinal struts 608, as described in further detail below. One of the plurality of longitudinal struts 608 may be stent spine 610, or alternatively, stent spine 610 may be affixed to one of the plurality of longitudinal struts 608.

As shown in FIG. 13B, stent 604 further may include proximal atraumatic section 614 at proximal end 601 of stent 604, and distal atraumatic section 616 at distal end 603 of stent 604. Distal atraumatic section 616 may comprise a closed cell structure formed by one or more circumferentially-extending struts coupled to and extending from the distal-most strut 606. The circumferentially-extending struts forming distal atraumatic section 616 may be thinner than the struts of struts 606, to thereby provide a significantly less stiff section than the main stent structure formed by circumferentially-extending struts 606 and longitudinal struts 608. Moreover, the closed structure (e.g., no exposed cell apex) of distal atraumatic section 616 generates a gradual transition interaction between the vessel wall and the stent main structure, thereby preventing a draping effect and risk of vessel perforation. Similarly, proximal atraumatic section 614 also may comprise a closed cell structure formed by one or more circumferentially-extending struts coupled to and extending from the proximal-most strut 606, which may also be thinner than the struts of struts 606 to thereby provide a significantly less stiff section than the main stent structure. Due to the weak structure of proximal atraumatic section 614, the axial force generated by the radial compression of proximal atraumatic section 614 may be negligible. Moreover, proximal atraumatic section 614 similarly provides a soft transition for the vessel interaction, which further allows for significant variation in oversizing of stent 604.

In addition, circumferentially-extending struts 606 may each have a sinusoidal or zig-zag or otherwise oscillating circumferential pattern. For example, as shown in FIG. 13B, struts 606 may include an alternating pattern of valleys 605 and apexes 607. Longitudinal struts 608 may be selectively positioned to connect adjacent struts 606, while maintaining the overall structure of stent 604 in the axial direction and controlling the radial force applied by stent 604 onto the vessel, as well as providing axial integrity for recapture. Preferably, longitudinal struts 608 are only coupled to and extend between select valleys 605 of circumferentially-extending struts 606, such that apexes 607 are “unsupported” by longitudinal struts 608. The unsupported apexes 607 of each cell of stent 604 may extend unidirectionally in the same direction, e.g., facing distally in the inferior direction of the SVC (e.g., in the direction of blood flow towards the heart) against the direction of migration of stent 604 due to the crawling effect described above. Accordingly, as stent 604 compresses radially once deployed in the vessel, unsupported apexes 607 may flare radially outward, as shown in FIG. 13A, and interact with the inner wall of the vessel to thereby provide retention forces against inferior migration of stent 604. In addition, the direction of unsupported apexes 607 may facilitate recapture of stent 604 as they will fold radially inward as the recapturing sheath is pushed distally relative to stent 604 (e.g., in a direction from proximal end 601 toward distal end 603).

Moreover, as longitudinal struts 608 do not connect each adjacent valley 605 of struts 606, each cell of stent 604 between adjacent struts 606 without a longitudinal strut 608 connecting adjacent valleys 605 within the cell defines a local open cell, e.g., open cells 612 of stent 604. Open cells 612 allow for less axial rigidity locally when the patient's anatomy interacts with stent 604, thereby providing localized lateral flexibility and improvement of conformability with the vessel. The position of open cells 612 may alternate radially along the circumference of stent 604 to preserve axial rigidity of stent 604. For example, preferably, no two open cells are adjacent along the circumference of stent 604. In addition, at least one longitudinal strut 608 may extend along the entire length of stent 604 (excluding the proximal and distal atraumatic sections 614, 616 described in further detail below). For example, preferably, longitudinal struts 608 adjacent to anchor support 602, and accordingly anchor tube 360, may extend from the distal-most strut of struts 606 to the proximal-most strut of struts 606, to thereby reduce the mechanical stress from fatigue on the stent tube welds and the mechanical stress due to motion of anchor tube 360.

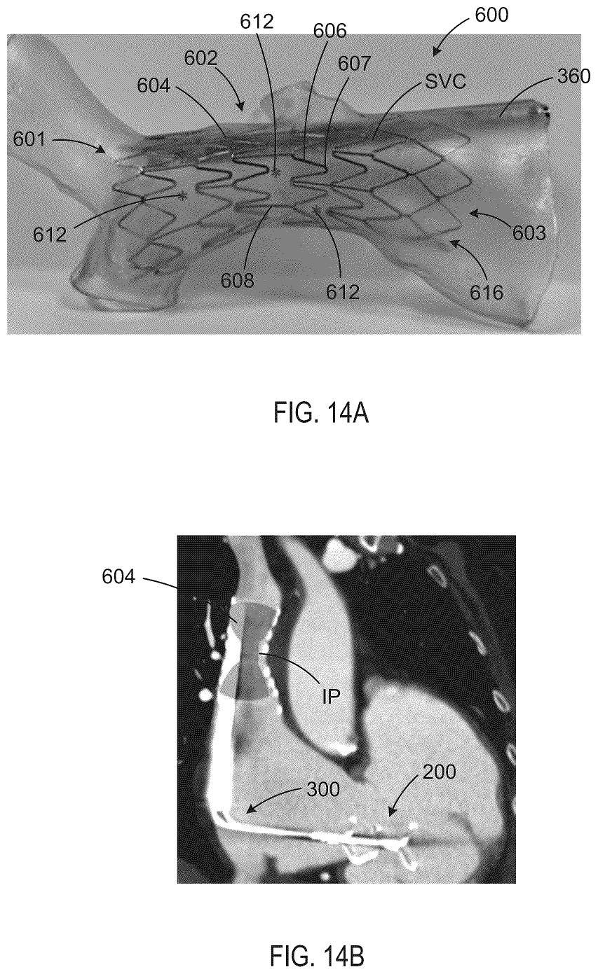

By not including longitudinal struts 608 at discrete locations on stent 604, e.g., within the middle region of stent 604, flexibility and conformability of stent 604 may be improved within the anatomy. Accordingly, the conformability of the stent itself may provide an antimigration feature. For example, as shown in FIGS. 14A and 14B, due to the vessel inflection point IP created by the patient's anatomy, the conformability of stent 604 results in a bow tie shape with stent 604 having a smaller outer diameter at vessel inflection point IP and larger outer diameters above and below vessel inflection point IP, thereby making stent 604 more resistant to migration.

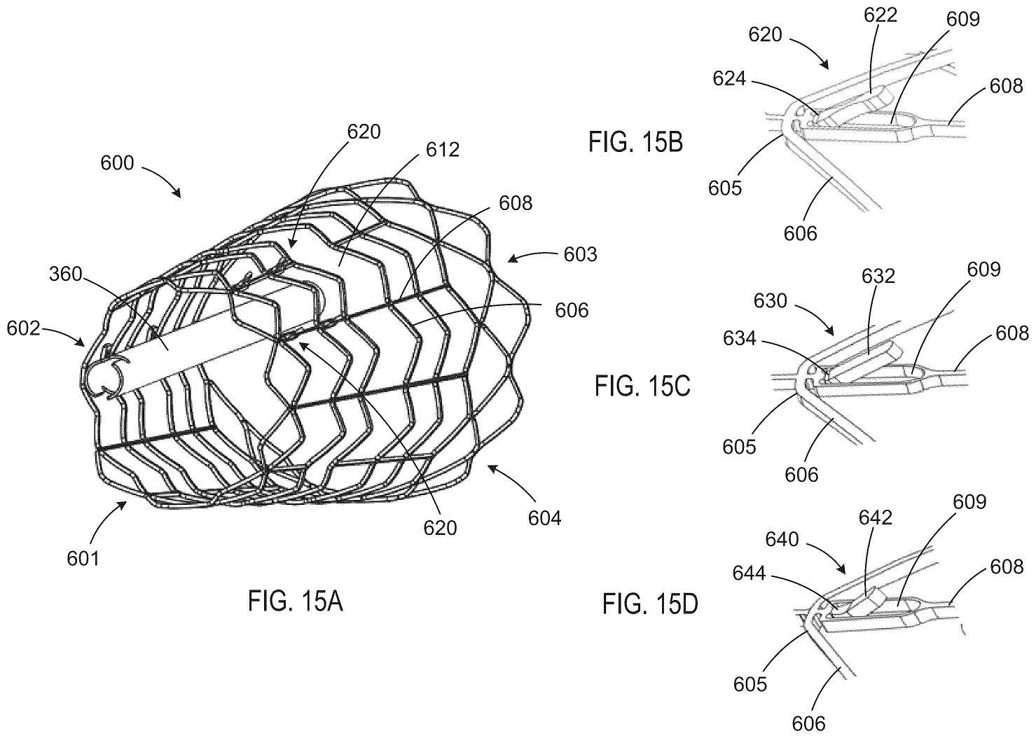

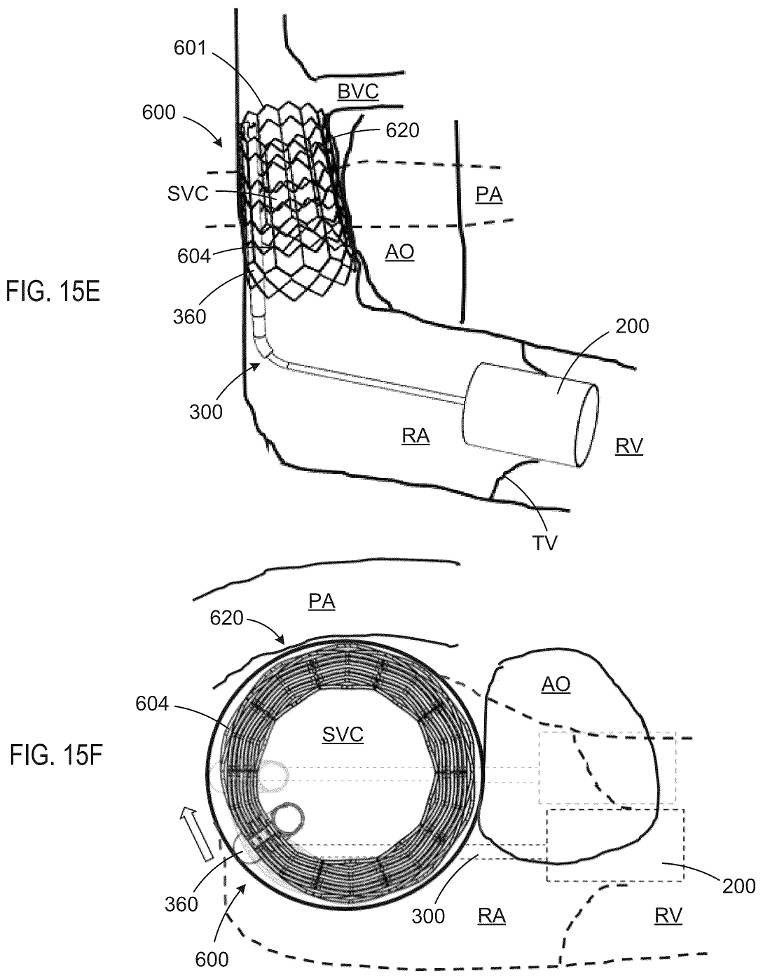

Referring now to FIGS. 15A to 15D, various short flare antimigration features of the stent are provided. As shown in FIG. 15A, at least the proximal region of stent 604, e.g., adjacent to proximal end 601 of stent 604, may include a plurality of expandable short flares 620. Short flares 620 may be formed as part of longitudinal struts 608, and may be configured to transition between a collapsed state and an expanded, deployed state where short flares 620 extend outwardly from the outer surface of stent 604 in the distal direction, preferably at an angle selected to ensure that the interaction between short flare 620 with the inner layer of the vessel is sufficient to provide retention, while preventing trauma to the vessel and risk of sheath delamination upon deployment stent 604, and accordingly short flares 620.

As shown in FIG. 15B, short flare 620 may include flare 622 pivotally coupled to longitudinal strut 608 via hinge 624, such that flare 622 may self-expand from a collapsed position where flare 622 is disposed within opening 609 defined within longitudinal strut 608, to an expanded, deployed state, where flare 622 extends radially outward at an angle relative to longitudinal strut 608 in the distal direction, thereby providing a reaction force against the vessel in a direction opposite to the direction of migration forces of stent 604. Accordingly, in the expanded, deployed state, short flares 620 may generate a localized pressure point within the inner layer of the vessel in which stent 604 is deployed. Moreover, the distal tip of flare 622 may be rounded to reduce the risk of vessel perforation and sheath delamination upon stent deployment. As shown in FIG. 15B, flare 622 may comprise a dumbbell-shaped profile configured to allow for additional flexibility of flare 622 to further reduce the risk of delamination of the delivery sheath by flare 622 during stent delivery.

FIGS. 15C and 15D illustrate alternatively configurations of the short flare antimigration features. As shown in FIG. 15C, short flare 630 may be constructed similar to short flare 620 of FIG. 15B. For example, short flare 630 may include flare 632 pivotally coupled to longitudinal strut 608 via hinge 634, such that flare 632 may self-expand from a collapsed position where flare 632 is disposed within opening 609 defined within longitudinal strut 608, to an expanded, deployed state, where flare 632 extends radially outward at an angle relative to longitudinal strut 608 in the distal direction, thereby providing a reaction force against the vessel in a direction opposite to the direction of migration forces of stent 604. Unlike flare 622, flare 632 may not have a dumbbell-shaped profile. Instead, flare 632 may comprise a straight, linear profile, and therefore may be stiffer than flare 622. As shown in FIG. 15D, short flare 640 may be constructed similar to short flare 640 of FIG. 15C. For example, short flare 640 may include flare 642 pivotally coupled to longitudinal strut 608 via hinge 644, such that flare 642 may self-expand from a collapsed position where flare 642 is disposed within opening 609 defined within longitudinal strut 608, to an expanded, deployed state, where flare 642 extends radially outward at an angle relative to longitudinal strut 608 in the distal direction, thereby providing a reaction force against the vessel in a direction opposite to the direction of migration forces of stent 604. As shown in FIG. 15D, flare 642 may have shorter length than flare 632.

As shown in FIGS. 15E and 15F, when implanted within superior vena cava SVC, stent tube 360 apposes towards the lateral atrial wall of the SVC. FIG. 15F illustrates a cranial view of stent 604 within superior vena cava SVC. The angular placement of stent tube 360 along the main axis of the SVC may be controlled during implantation, and may be selected within a predefined range. As described above, proximal end 601 of stent 604 may be accurately positioned relative to the BCV confluence, e.g., without extending completely across the confluence, as shown in FIG. 15E. Accordingly, the antimigration features of stent 604 (e.g., short flares 620, 630, 640) may disposed at predefined positions along stent 604 (e.g., toward the proximal region of stent 604) such that, within the predefined range of angular placement of stent tube 360 along the main axis of the SVC and based on the distance between brachiocephalic vein BCV and pulmonary artery PA, these antimigration features may always be positioned above pulmonary artery PA and/or aorta AO towards the media sternal area to thereby prevent any potential interaction with pulmonary artery PA and/or aorta AO. Accordingly, short flares 620, 630, 640 allow for systematic positioning away from the aorta.

Additionally, or alternatively, the antimigration features of stent 604 (e.g., short flares 620, 630, 640) may selectively disposed at predefined radial positions along a circumference of stent 604, such that, when stent 604 is deployed within the SVC, the antimigration features are disposed along stent 604 at radial positions away from the medial portion of the patient (e.g., away from aorta AO). For example, when stent tube 360 apposes towards the lateral atrial wall of the SVC, no antimigration features may be disposed on the radial region of stent 604 opposite stent tube 360, to thereby minimize contact with aorta AO and avoid risk of perforation/bleeding of aorta AO. Accordingly, if the radial position of stent tube 360 along stent 604 is twelve o'clock, there may be no antimigration features disposed along stent 604 at the radial positions of at least 5 o'clock, 6 o'clock, or 7 o'clock relative to stent tube 360.

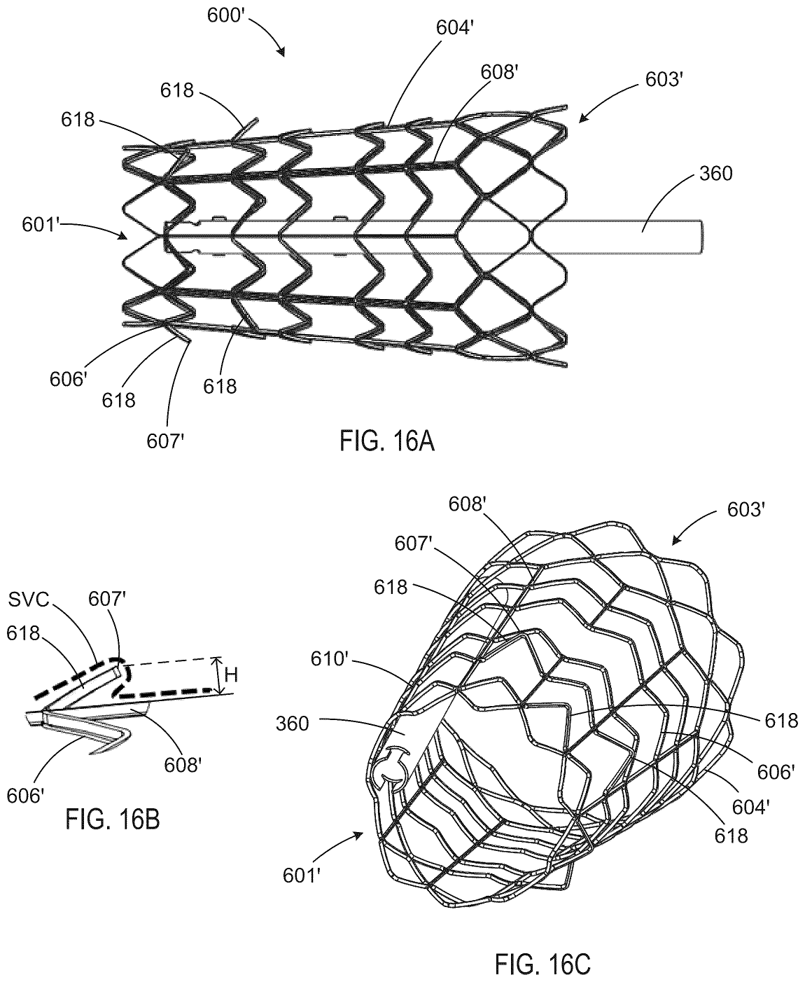

Referring now to FIGS. 16A to 16C, various flared cell antimigration features of stent 604′ of anchor 600′ are provided. Stent 604′ of anchor 600′ may be constructed similar to stent 604 of anchor 600, with similar components having like-prime reference numerals. For example, stent 604′ may be formed by a plurality of circumferentially-extending struts 606′, selectively interconnected via a plurality of longitudinal struts 608′, and further may be coupled to anchor tube 360 via stent spine 610′. Stent 604′ differs from stent 604 in that, in addition to, or instead of short flare antimigration features, stent 604′ may include a plurality flared cells 618. Flared cells 618 may be formed from the cells of stent 604′ where unsupported apexes 607′ of the cells are configured to deflect outwards in its set position. For example, as shown in FIG. 16A, unsupported apexes 607′ of flared cells 618 may be configured to deflect radially outward, e.g., upon radial compression of stent 604′, in the distal direction (e.g., in the direction of blood flow towards the heart) at an angle (e.g., greater than the outward flex of the other unsupported apexes, as described above), thereby providing a reaction force against the vessel in a direction opposite to the direction of migration forces of stent 604′.

The angle of deflection of flared cells 618 may preferably be selected to ensure that the interaction between flared cells 618 with the inner layer of the vessel is sufficient to provide retention, while preventing trauma to the vessel and risk of sheath delamination upon deployment stent 604′, and accordingly flared cells 618. Accordingly, in the deflected state, flared cells 618 may generate a localized pressure point within the inner layer of the vessel in which stent 604′ is deployed. For example, as shown in FIG. 16B, the flare height H of apex 607′ of flared cell 618, e.g., relative to longitudinal strut 608′, may be selected and controlled to sufficiently permit the inner wall of the SVC to wrap around at least apex 607′ of flared cell 618, to thereby provide a resisting force to migration of stent 604′, without applying excessive pressure onto the vessel wall. Moreover, apexes 607′ of flared cells 618 may be rounded to reduce the risk of vessel perforation and sheath delamination upon stent deployment.

Like the short flare antimigration features, flared cells 618 may be disposed at predefined positions along the proximal region of stent 604′ such that they may similarly always be positioned above pulmonary artery PA and/or aorta AO towards the mediastinal area to thereby prevent any potential interaction with pulmonary artery PA and/or aorta AO. Accordingly, flared cells 618 allow for systematic positioning away from the aorta. In addition, like the short flare antimigration features, flared cells 618 may be selectively disposed at predefined radial positions along the circumference of stent 604′ such that they may similarly always be positioned away from the medial portion of the patient (e.g., away from aorta AO), to thereby minimize contact with aorta AO and avoid risk of perforation/bleeding of aorta AO. Moreover, the position of flared cells 618 may alternate radially along the circumference of stent 604′ to provide maximum localized interaction with the vessel for better retention in the vessel, as shown in FIG. 16C. Preferably, flared cells 618 are not positioned adjacent to stent tube 360.

In addition, at least some or all of the cells adjacent to, e.g., bordered by, longitudinal struts 608′ may comprise a flared apex having a low flaring angle, e.g., less than flare height H shown in FIG. 16B, to thereby impact the contact angle of the flared apex with the SVC wall and, therefore, generate an additional resistance to migration while reducing the risk of vessel wall perforation. In order to treat a wider population, as will be understood by a person having ordinary skill in the art, the same stent architecture may be applied to stents with different diameters, e.g., in the expanded deployed state. For example, as the radial stiffness of the stent may be defined by the circumferential number of strut cells per strut row, e.g., “columns,” as well as the thickness of the stent wall, to preserve the radial stiffness, larger diameter stents preferably include a higher number of columns and/or have thicker stent walls. Moreover, as the stiffness of the flared cell for providing additional retention may be a function of overall structure of the cell design of the stent and/or the thickness of the stent wall, for larger diameter stents, the thickness of the stent wall may be increased to provide a flare stiffness sufficient to provide the required pressure against the vessel for increased retention within the vessel.

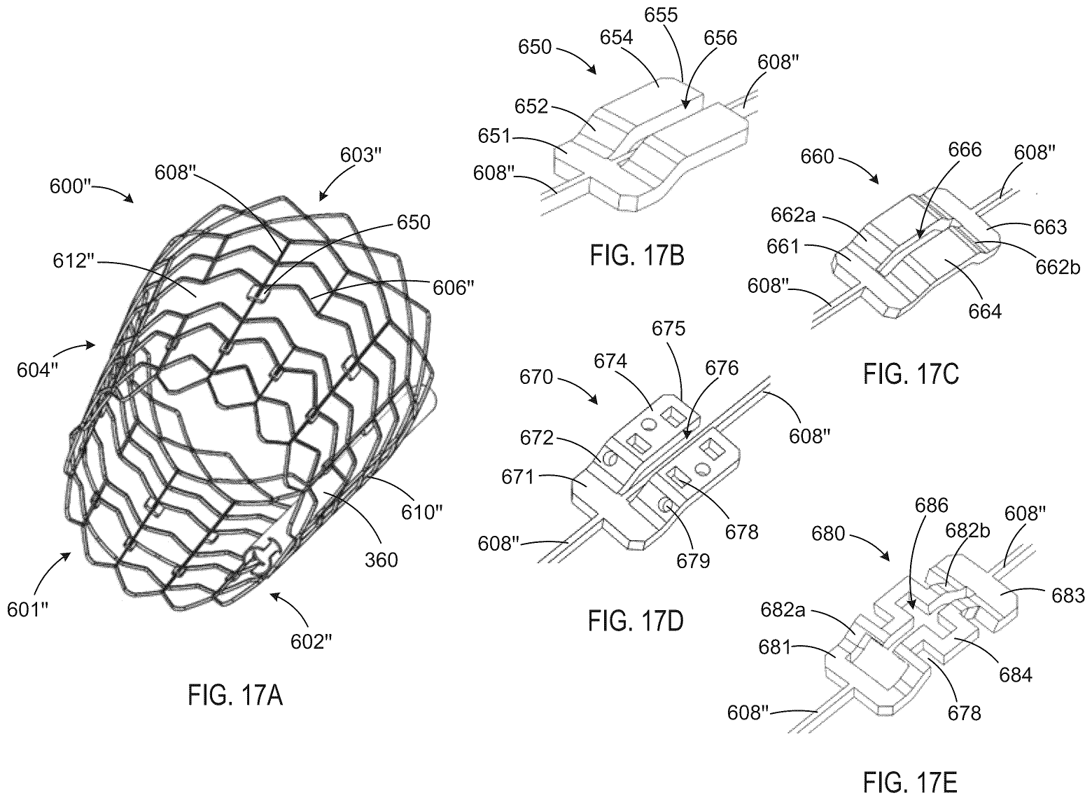

Referring now to FIGS. 17A to 17D, an exemplary anchor having friction pads is provided. Anchor 600″ may be constructed similar to anchor 600, 600′ with similar components having like-double prime reference numerals. For example, anchor 600″ may comprise stent 604″, which may be formed by a plurality of circumferentially-extending struts 606″, selectively interconnected via a plurality of longitudinal struts 608″, and further may be coupled to anchor tube 360 via stent spine 610″. Stent 604″ differs stent 604, 604′ in that, instead of, or in addition to, short flare and/or flared cell antimigration features, stent 604″ may comprise one or more antimigration friction pads 650 disposed on one or more longitudinal struts 608″, as shown in FIG. 17A. Friction pads 650 may be selectively positioned at different locations over stent 604″ where sufficient width between the supporting struts and surrounding cells allows the respective friction pads to be positioned in a crimped position, e.g., when stent 604″ is in its collapsed delivery state. FIG. 17A illustrates exemplary sizing and positioning of friction pads 650, selected to facilitate stent crimping while maximizing the density of the friction pads. Friction pads 650 may be formed integrally with stent 604″, e.g., as part of the laser cutting pattern of stent 604″. Friction pads 650 may be disposed on the proximal, e.g., trailing, region of stent 604″, as shown in FIG. 17A.

As shown in FIG. 17B, friction pads 650 may each comprise raised portions 654 coupled to longitudinal strut 608″, e.g., via flat portion 651 and ramp portions 651. For example, each friction pad 650 may comprise flat portion 651 coupled to and extending laterally from longitudinal strut 608″, and raised portions 654 raised above flat portion 651 via ramp portions 652, such that raised portions 654 comprise a height above longitudinal strut 608″ and flat portion 651. In some embodiments, raised portions 654 of friction pad 650 may be at least partially flared to only slightly raise the distal end of raised portions 654 about the longitudinal strut 608″ distal to friction pad 650. As shown in FIG. 17B, friction pad 650 comprises a width greater than the width of longitudinal strut 608″, and ramp portions 652 may extend upward from flat portion 651, preferably at an angle, on both sides of longitudinal strut 608″, thereby defining an opening, e.g., track 656 aligned with longitudinal strut 608″, along ramp portions 652 and raised portions 654, e.g., between distal edges 655 of friction pad 650 and the connection point of ramp portions 652 and flat portion 651. Alternatively, in some embodiments, raised portion 654 and ramp portion 651 may extend from flat portion 651 on only one side of longitudinal strut 608″.

Moreover, as raised portions 654 are raised above longitudinal strut 608″, e.g., further radially outward from the longitudinal axis of stent 604″, distal edges 655 provide additional contact against the inner wall of the blood vessel, e.g., the SVC, in a direction opposite the potential migration direction. In addition, at least raised portions 654 of friction pads 650 may be textured via a surface roughening process (e.g., bead blasting with masking, laser etching, ablation, etc.) to increase the friction forces between the stent and the SVC at the friction pad locations. For example, this friction may be increased with the localised pressure provided via the raised sections of the friction pad. In addition, the texturing may be applied on the friction pads only so as to not impact the fatigue resistance of the stent. In some embodiments, the friction pads may not include raised portions, and accordingly ramp portions, such that the textured surface areas of the friction pad may be radially aligned with the longitudinal struts, thereby facilitating case of manufacture.

Referring now to FIG. 17C, an alternative friction pad with interrupted longitudinal struts is provided. Friction pad 660 may be constructed similar to friction pad 650. For example, friction pad 660 may include proximal flat portion 661 extending laterally from longitudinal strut 608″, and raised portions 664 extending upwardly from proximal flat portion 661, preferably at an angle, via ramp portions 662, and defining track 666. Like raised portions 654, at least raised portions 664 of friction pads 660 may be textured to thereby increase the friction forces between the stent and the SVC at the friction pad locations. Friction pad 660 differs from friction pad 650 in that the distal end of friction pad 660 may be coupled to longitudinal strut 608″ via distal flat portion 663 extending laterally from longitudinal strut 608″ at location distal to proximal flat portion 661 and raised portions 664, as shown in FIG. 17C. For example, raised portions 664 may be raised above distal flat portion 663 via ramp portions 662.

As shown in FIG. 17C, longitudinal strut 608″ may be interrupted, e.g., may not extend across track 666 defined by raised portions 664 and at least a portion of ramp portions 662. Accordingly, the opening middle section of friction pad 660 defined by track 666 reduces impact of the stiffness of longitudinal strut 608″, such that the stiffness of longitudinal strut 608″ is not impacted via inclusion of the friction pad and, thus, prevents friction pad 660 from impacting the stent's conformability to the vessel wall. The size and shape of track 666, e.g., the width and length of track 666, may be selected to tune the impact of the friction pad on the stiffness of the longitudinal strut versus the amount of additional surface area available for texturing, e.g., raised portions 664.

Referring now to FIG. 17D, an alternative friction pad with an increased number of antimigration edges is provided. Friction pad 670 may be constructed similar to friction pad 650. For example, friction pad 670 may include flat portion 671 extending laterally from longitudinal strut 608″, and raised portions 674 having distal edges 675 and extending upwardly from flat portion 671, preferably at an angle, via ramp portions 672, and defining track 676. Friction pad 670 differs from friction pad 650 in that friction pad 670 may comprise one or more openings disposed on at least raised portions 674 and, optionally, on ramp portions 672, to thereby provide additional edges that contact against the inner wall of the blood vessel, e.g., the SVC, in a direction opposite the potential migration direction. As shown in FIG. 17D, friction pad 670 may include one or more rectangular shaped opening 678 and one or more circular shaped openings 679. As will be understood by a person having ordinary skill in the art, the openings may have a shape other than a rectangular and/or circular shape. Moreover, in some embodiments, like raised portions 654, at least raised portions 674 of friction pads 670 may be textured to thereby increase the friction forces between the stent and the SVC at the friction pad locations.

Referring now to FIG. 17E, an alternative friction pad with interrupted longitudinal struts and an increased number of antimigration edges is provided. Friction pad 680 may be constructed similar to friction pad 660. For example, friction pad 680 may include proximal flat portion 681 and distal flat portion 683 extending laterally from longitudinal strut 608″, and raised portions 684 extending upwardly from proximal flat portion 671 and distal flat portion 683, preferably at an angle, via proximal ramp portion 682a and distal ramp portion 682b, respectively, and defining track 686. Friction pad 680 differs from friction pad 660 in that raised portions 684 and/or track 686 may comprise a geometry defining a plurality of distal edges to thereby provide additional edges that contact against the inner wall of the blood vessel, e.g., the SVC, in a direction opposite the potential migration direction while providing compliance to longitudinal struts 608″, e.g., conformability to the anatomy. As shown in FIG. 17E, raised portions 684 may comprise a plurality of gaps 678 extending laterally inward and positioned between raised portions 684 and ramp portions 682, each gap 678 defining a distal edge configured to contact against the inner wall of the blood vessel in a direction opposite the potential migration direction. Moreover, as shown in FIG. 17E, the portion of track 686 disposed on raised portions 684 and/or the portion of track 686 disposed on proximal ramp portion 682a may have a larger width than the portions of track 686 extending adjacent gaps 678 between raised portions 684 and proximal and distal flat portions 681, 683, thereby further providing additional distal edges to contact against the inner wall of the blood vessel in a direction opposite the potential migration direction. For example, the larger width track 686 extending along raised portions 684 may further provide a pinching type interaction with the vessel wall, which may further improve the resistance to migration.

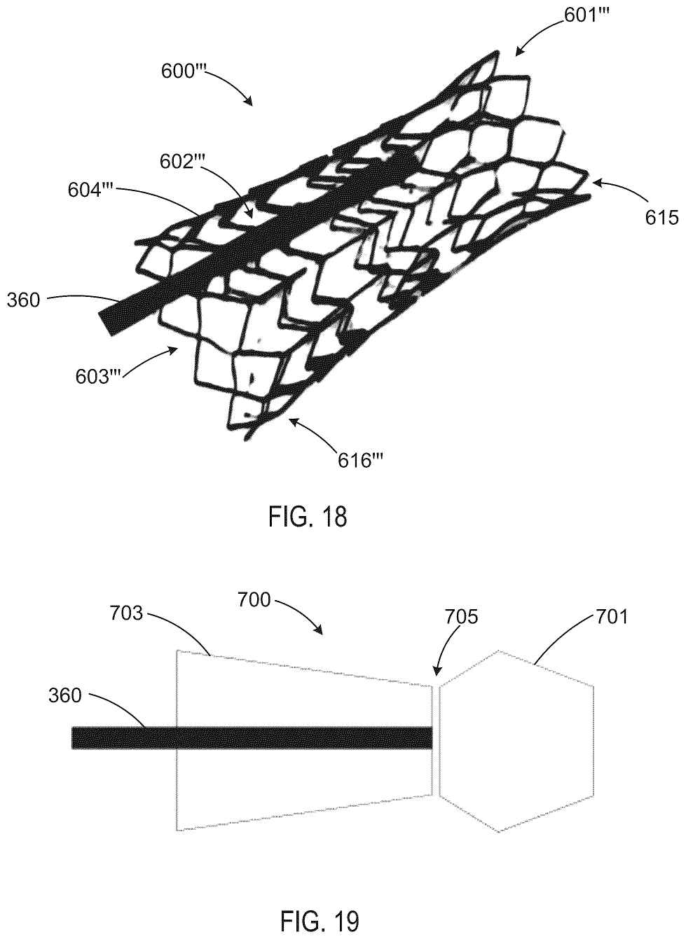

Referring now to FIG. 18, an exemplary anchor having a flared cranial end region is provided. Anchor 600′″ may be constructed similar to anchor 600, 600′, 600″ with similar components having like-double prime reference numerals. Anchor 600′″ differs from anchor 600, 600′, 600″ in that the proximal/cranial end of stent 604′″ of anchor 600′″, e.g., adjacent to proximal end 601′″ of stent 604′″, may comprise flared cranial end region 615. Flared cranial end region 615 may be configured to flare radially outward from a central axis of stent 600′″ in the expanded, deployed state, such that stent 604′″ may be selectively designed to conform to the specific anatomy in the region of the right heart SVC. For example, stent 604′″ may use the confluence of the SVC to the left and right brachiocephalic veins as the anchor region, such that, when stent 604′″ is implanted within the SVC, flared cranial end region 615 may be deployed adjacent the brachiocephalic vein confluence and open into the confluence of the left and right brachiocephalic veins. This feature allows stent 604′″ to expand into this region, thereby providing an outer diameter at cranial end 601′″ that is larger than the outer diameter of stent 604′″ in the direction from the confluence towards the SVC, which results in a stent design that aids in migration resistance. Particularly, since stent 604′″ needs to be “pulled” down the SVC and reduced in diameter, the radial resistance force of stent 604′″ to the reduction in diameter provides for a mechanical anchor of stent 604′″ in the SVC.

Referring now to FIG. 19, an exemplary anchor having a high deflection decoupling section is provided. Anchor 700 may be constructed similar to anchors 500, 600, 600′, 600″ such that anchor 700 may be coupled to anchor tube 360 and configured to be implanted within a blood vessel (e.g., the SVC) to maintain the prosthetic device suspended within the native valve leaflets. Unlike anchors 500, 600, 600′, 600″, anchor 700 may include a stent structure having proximal anchoring region 701, distal main stent body 703, and decoupling region 705 disposed between and connecting anchoring region 701 and main stent body 703. Accordingly, distal main stent body 703 may be flexibly decoupled from main stent body 703 via decoupling region 705, which allows the stent structure of anchor 700 to have a dual function. For example, main stent body 703 may provide support to stent tube 360, which guides placement of the prosthetic device within the native heart valve, and anchoring region 701 may anchor the stent structure within the target blood vessel.

As shown in FIG. 19, main stent body 703 may have a tapered profile such that the cross-sectional area of main stent body 703 increases from a proximal end of main stent body 703 in a distal direction towards the distal end of main stent body 703. Moreover, anchoring region 701 may comprise a tapered profile in a direction opposite to that of main stent body 703, such that at least a portion of anchoring region 701 has a cross-sectional area that increases from the distal end of anchoring region 701 in a proximal direction towards the proximal end of anchoring region 701, thereby providing a counter-balance loading direction to main stent body 703 to aid in migration resistance of anchor 700. Moreover, decoupling region 705 may be configured to aid migration resistance of anchor 700 as it limits transmission of the movements on the support, e.g., support 300 (not shown) coupled to anchor tube 360, to anchoring region 701.

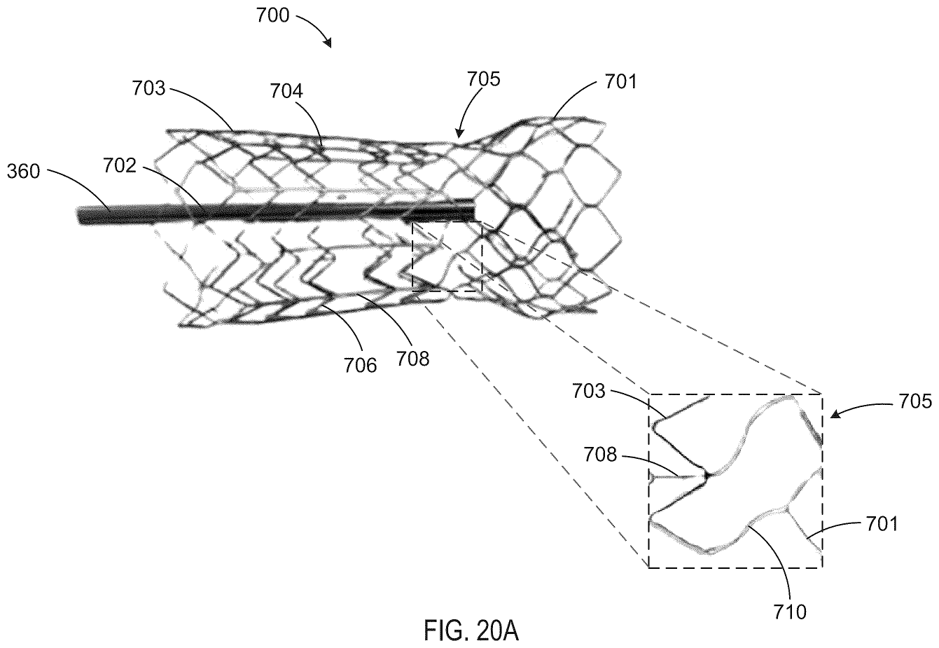

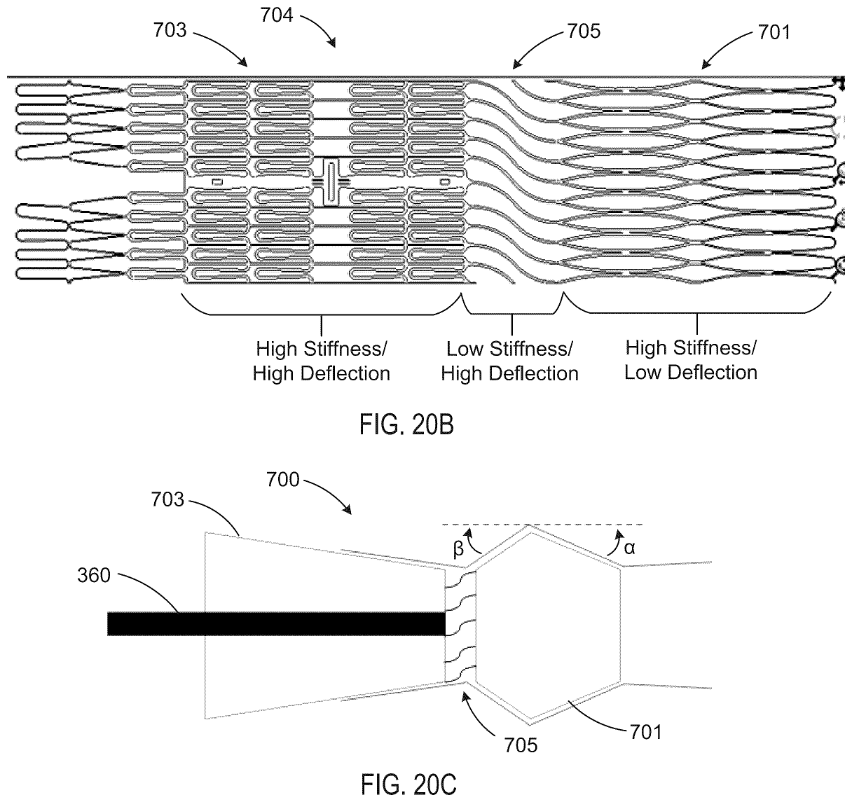

FIGS. 20A to 20C illustrate an exemplary anchor having a decoupling section formed by wavy struts of the frame of the stent of the anchor. As shown in FIG. 20A, stent 704 of anchor 700 may include decoupling region 705 formed by a plurality of wavy struts 710 extending between and connecting the distal end of anchoring region 701 with the proximal end of main stent body 703. For example, wavy struts 710 may extend in a generally longitudinal direction (e.g., in an S-shape) between the proximal-most strut of main stent body 703 and the distal-most strut of anchoring region 701. FIG. 20B illustrates the frame of stent 704 of anchor 700. As shown in FIG. 20B, main stent body 703 (excluding any distal atraumatic region extending distally from the distal end of main stent body 703) comprises high stiffness and high deflection properties, decoupling region 705 comprises low stiffness and high deflection properties, and anchoring region 701 comprises high stiffness and low deflection properties. Due to the longer strut length of wavy struts 710 for a given axial length of stent 704, decoupling region 705 may be softer (e.g., more flexible), which decreases the strains, and accordingly stress, of stent 704, thereby reducing the translation of forces from main stent body 703 to anchoring region 701. For example, the stress in a stent may be calculated with the following equations:

$$ε = Z \frac{π ( Δ D )}{M} \frac{w}{{L}^{2}}$$

$$σ = \frac{F}{A}$$

$$σ = E ε ( Hooke ’ s Law )$$

Where ΔD is the difference between unconstrained and constrained diameter, w=strut width, t=strut thickness, L=length of strut, and M=circumferential number of strut cells per strut row, e.g., “columns,” Z=constant in superelastic Nitinol. See, for example, Pelton, A R et al. “Fatigue and durability of Nitinol stents.” Journal of the mechanical behavior of biomedical materials vol. 1,2 (2008): 153-64. doi: 10.1016/j.jmbbm.2007.08.001. Moreover, due to the increased oversizing on the cranial end of anchoring region 701, stent 704 may have the inclination to migrate atrially. However, as shown in FIG. 20C, If α<β the net force on anchor 700 will be in the cranial direction.

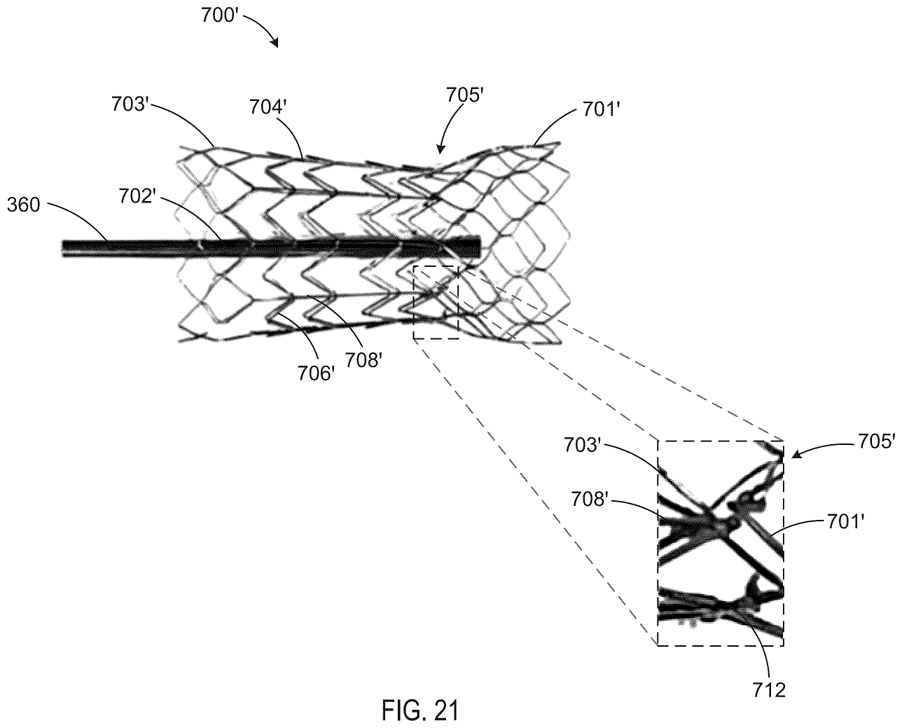

Referring now to FIG. 21, an alternative exemplary anchor having a decoupling section formed by suture attachments of the frame of the stent of the anchor is provided. Anchor 700′ may be constructed similar to anchor 700 with similar components having like-prime reference numerals. Anchor 700′ differs from anchor 700 in that decoupling region 705′ may be formed by suture attachments 712, such that suture attachments 712 extend between and connect the proximal-most strut of main stent body 703′ and the distal-most strut of anchoring region 701′. Suture attachments 712 are configured to reduce the translation of forces from main stent body 703′ to anchoring region 701′, to thereby to aid migration resistance of anchor 700′.

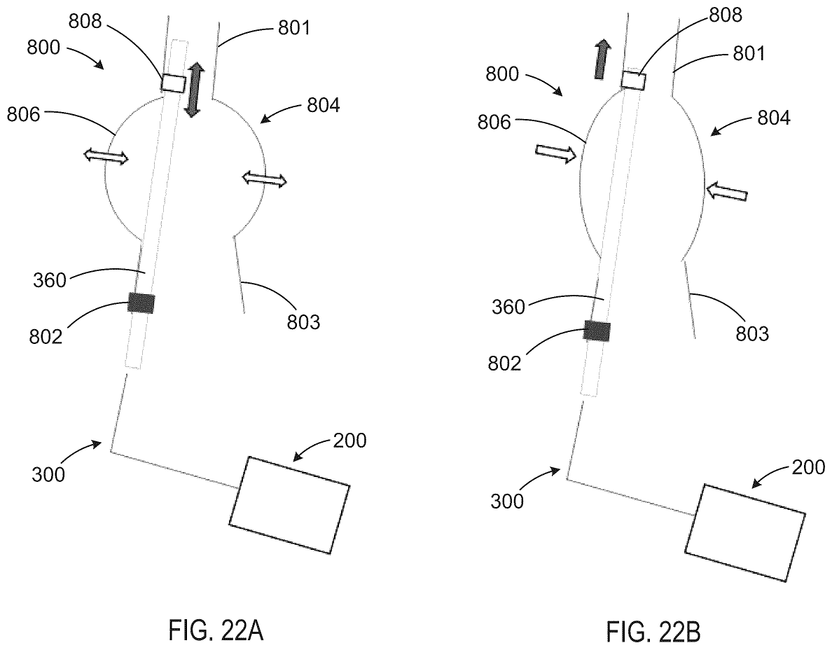

Referring now to FIGS. 22A and 22B, a selectively expandable anchor is provided. Anchor 800 may include stent 804 adjustably coupled to anchor tube 360 and configured to be implanted within a blood vessel (e.g., the SVC) to maintain the prosthetic device suspended within the native valve leaflets. As shown in FIGS. 22A and 22B, stent 804 has proximal/cranial region 801, distal region 803, and selectively expandable middle region 806 disposed between proximal region 801 and distal region 803, and anchor tube 360 may extend at least partially within proximal region 801 of stent 804. Distal region 803 of stent 804 may be fixedly coupled to a distal portion of anchor tube 360 via fixed ring 802, and proximal region 801 may be slidably and lockably coupled to a proximal portion of anchor tube 360 via sliding ring 808. For example, sliding ring 808 may be fixed to proximal region 801, and configured to slide relative to anchor tube 360.

Accordingly, when anchor tube 360 is operatively connected to the actuator system, e.g., actuator 108, the actuator system may be actuated to axially move sliding ring 808, and accordingly, proximal region 801 fixed thereto, relative to anchor tube 360 while distal region 803 remains fixed relative to anchor tube 360 via fixed ring 802, which allows middle region 806 of stent 804 to transition between an expanded state as shown in FIG. 22A (e.g., when sliding ring 808 is displaced atrially/distally) and a contracted state as shown in FIG. 22B (e.g., when sliding ring 808 is displaced cranially/proximally). By collapsing middle region 806 of stent 804 towards the contracted state, stent 804 may be fully recaptured post-deployment, as well as repositioned within the blood vessel. The control of the displacement of sliding ring 808 may allow for different levels of stent oversizing for different anatomies. Once stent 804 is positioned and sized as intended, sliding ring 808 may be locked onto anchor tube 360 tube, e.g., via a friction lock mechanism, threaded interaction, radial clamping, etc. Accordingly, anchor tube 360 and sliding ring 808 may then be released from the actuator system, such that they may remain implanted along with anchor 800 and the prosthetic device.

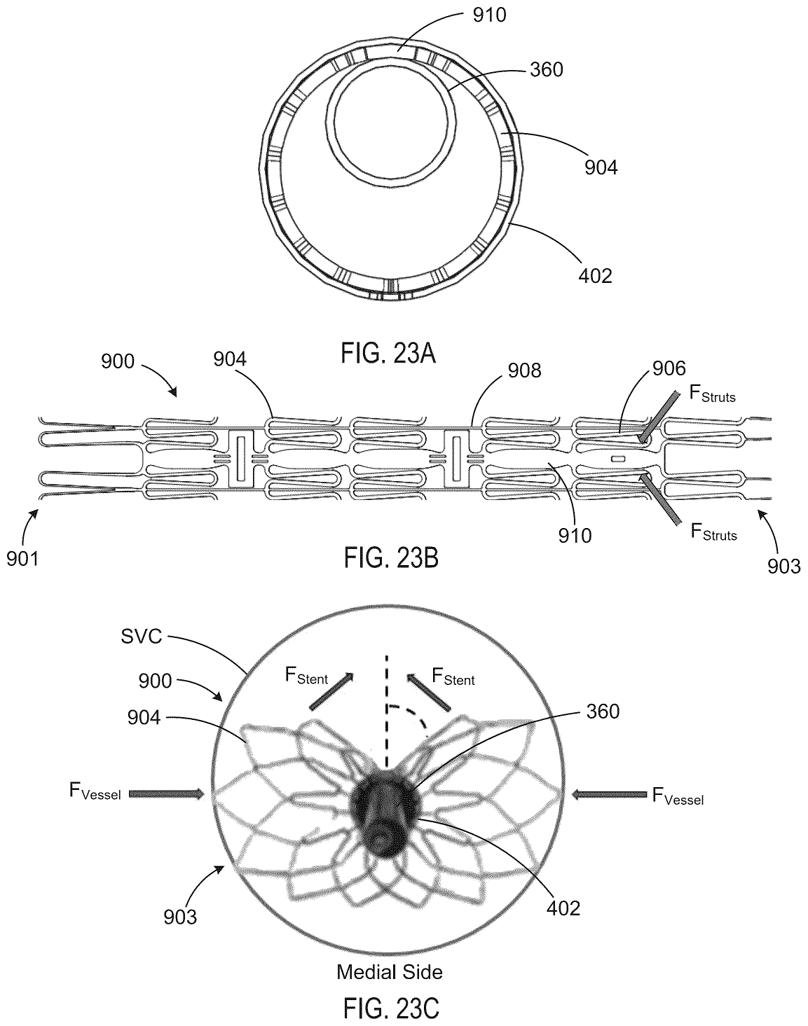

Referring now to FIGS. 23A to 23C, potential in-folding of an expandable anchor during deployment from a delivery sheath is described. Anchor 900 may comprise stent 904, which may be constructed similar to any of the stents described herein, e.g., stents 504, 604, 604′, 604″, 704, etc. For example, stent 904 may be formed by a plurality of circumferentially-extending struts 906, selectively interconnected via a plurality of longitudinal struts 908, and may be coupled to stent tube 360 via stent spine 910. As stent tube 360 is coupled to one side of the stent 904, when stent 904 is crimped in its collapsed delivery configuration within delivery sheath 402, stent tube 360 may not be centered within delivery sheath 402, as shown in FIG. 23A. For example, as shown in FIG. 23B, certain forces F_{Struts} may be applied to stent spine 910 and, accordingly, stent tube 360, by circumferentially-extending struts 906 when stent 904 is in its collapsed delivery configuration within delivery sheath 402, thereby forcing stent tube 360 towards the center of delivery sheath 402. Such forces F_{Struts} may cause at least some circumferentially-extending struts 906 adjacent stent spine 910 to move towards and thereby overlap with stent spine 910, as shown in FIG. 23B.

Moreover, when a large stent is used, when there is a lack of one or more longitudinal struts 908 in the middle region of the stent, when the stent has small wall thickness, and/or when the patient's specific anatomy biases stent tube 360 close to the medial wall of the blood vessel, e.g., the SVC, during deployment from delivery sheath 402 within the SVC, there may be a risk that the lateral sides of stent 904 generates a relatively high contact force F_{Vessel} with the lateral sides of the SVC in comparison with the medial side of the SVC, as shown in FIG. 23C, which may potentially cause an uneven deployment of stent 904 from delivery sheath 402. For example, as shown in FIG. 23C, when stent 904 is crimped in its collapsed delivery configuration, lateral forces force F_{Vessel} applied to the lateral sides of stent 904 by the vessel wall during deployment of stent 904 may generate forces F_{Stent} against the portions of stent 904 adjacent to stent spine 910, thereby forcing stent tube 306 towards the medial side of the SVC and potentially propagating in-folding of stent 904 along its length during deployment from delivery sheath 402. In other words, the reaction forces F_{Stent} of the vessel wall against stent 904 during deployment of stent 904 may cause stent 904 to bend along the connection point between stent 904 and stent spine 910, e.g., when forces F_{Stent} are higher than the hinge force at the base of stent spine 910, thereby forming a heart shape (e.g., “infolding”), as shown in FIG. 23C, and promoting closing of the angle between longitudinal struts 908 and stent spine 910. Accordingly, in some embodiments, the anchors described herein may be configured to prevent or otherwise eliminate any risk of stent in-folding during deployment, as described in further detail below.

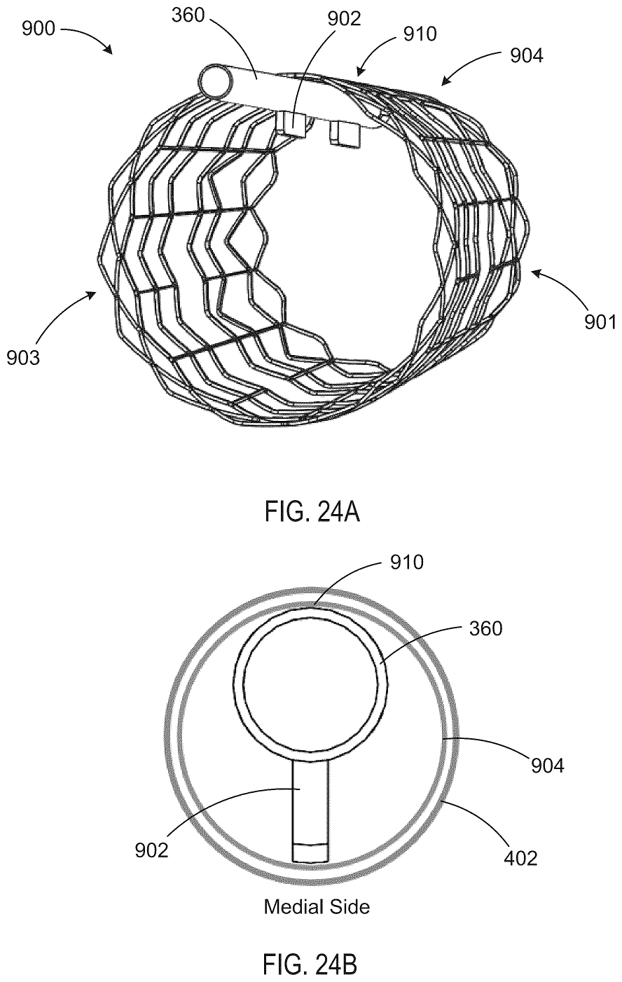

Referring now to FIGS. 24A and 24B, an exemplary expandable anchor having tilt control ribs configured to prevent potential stent in-folding during deployment is provided. As shown in FIG. 24A, stent tube 360 coupled to stent 904 of anchor 900 may comprise one or more tilt control ribs 902 extending laterally away from the outer surface of stent tube 360 along at least a portion of the length of stent tube 360. For example, tilt control ribs 902 may extend towards the central axis of stent 904 such that, when stent 904 is crimped in its collapsed delivery configuration within delivery sheath 402, as shown in FIG. 24B, tilt control ribs 902 may be sized and shaped to bias stent tube 360 towards the inner wall of delivery sheath 402. Accordingly, the free ends of tilt control ribs 902 may contact the portion of stent 904 opposite to stent spine 910 when stent 904 is in its collapsed delivery configuration within delivery sheath 402, to thereby force stent tube 360 against the inner wall of delivery sheath 402 and prevent stent tube 360 from deflecting towards the center of delivery sheath 402 and, accordingly, prevent the circumferentially-extending struts adjacent stent spine 910 from overlapping with stent spine 910.

Tilt control ribs 902 may be formed of the same material as stent tube 360, e.g., Nitinol, and may be welded to stent tube 360. Moreover, tilt control ribs 902 may be sized and shaped to reduce hemodynamic impact. For example, as shown in FIGS. 24A and 24B, tilt control ribs 902 may comprise a rectangular cross-section configured to minimize the impact on hemodynamic. As will be understood by a person having ordinary skill in the art, tilt control ribs 902 may comprise other cross-sectional shapes than a rectangle that also minimize the impact on hemodynamic. Additionally, while FIG. 24A shows tilt control ribs 902 formed of two discontinuous ribs, tilt control rib 902 may be formed of a single continuous rib extending along at least a portion of the length of stent tube 360, e.g., within the length of stent 904, or alternatively, may be formed of more than two discontinuous ribs.

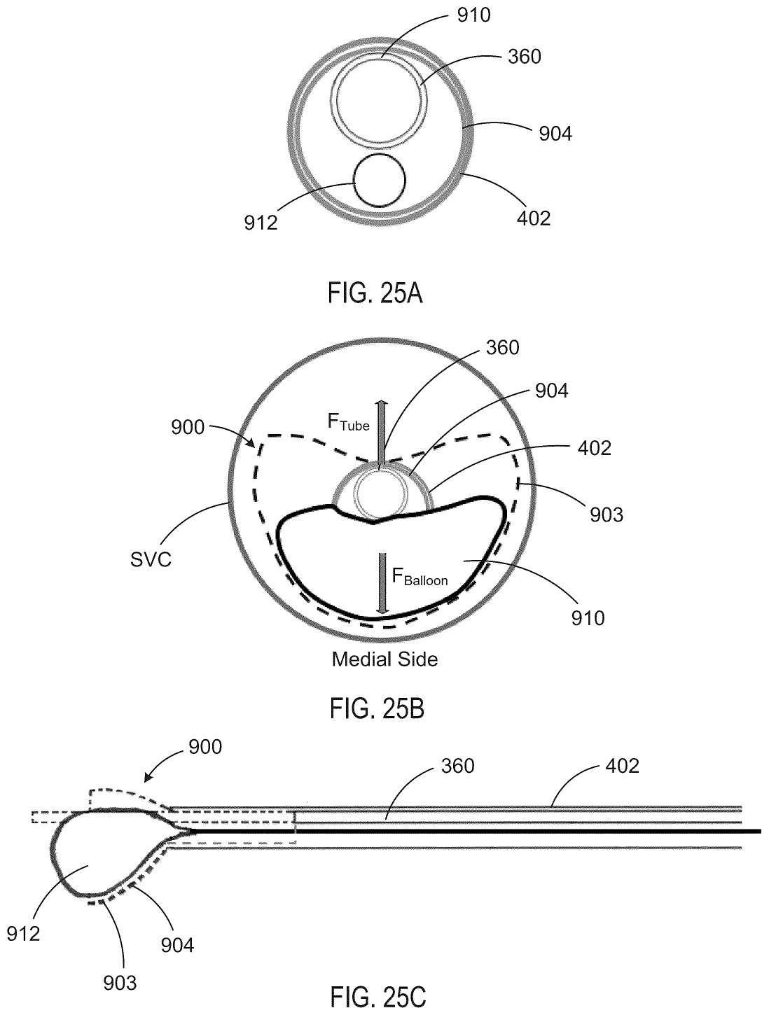

Referring now to FIGS. 25A to 25C, deployment of an exemplary expandable anchor via an exemplary balloon catheter to prevent potential stent in-folding during deployment is provided. As shown in FIG. 25A, balloon catheter 912 having an inflatable balloon may be disposed within delivery sheath 402 when stent 904 is crimped in its collapsed delivery configuration within delivery sheath 402, e.g., between the inner wall of stent 904 towards the medial side of the SVC and stent tube 360, to thereby force stent tube 360 against the inner wall of delivery sheath 402 and prevent stent tube 360 from deflecting towards the center of delivery sheath 402 and, accordingly, prevent the circumferentially-extending struts adjacent stent spine 910 from overlapping with stent spine 910. Accordingly, when stent 904 is partially deployed from delivery sheath 402, the balloon of balloon catheter 912 may be exposed beyond the distal end of delivery sheath 402, as shown in FIG. 25C.

As shown in FIG. 25B, inflation of the balloon of balloon catheter 912 as distal portion 903 of stent 904 is exposed from delivery sheath 402 may apply force F_{Balloon} against the portion of stent 904 opposite stent tube 360 towards the medial side of the SVC, as well as force F_{Stent} against stent tube 360, e.g., in a direction away from the medial side of the SVC, to thereby flatten stent 904 adjacent to stent tube 360, e.g., prevent bending of stent 904 along the stent spine 910, and facilitate even deployment of stent 904 from delivery sheath 402. Once stent 904 is fully deployed within the SVC, balloon catheter 912 may be deflated and retracted within delivery sheath 402. Accordingly, inflation and deflation of balloon catheter 912 may be controlled via an additional port into the sheath hub. Additionally, or alternatively, balloon catheter 912 may be threaded through a port in the sheath hub, and an inflation and deflation port may be disposed on balloon catheter 912.

In some embodiments, instead of balloon catheter 912, a cylindrical spacer tube may be disposed within delivery sheath 402 when stent 904 is crimped in its collapsed delivery configuration within delivery sheath 402, e.g., between the inner wall of stent 904 towards the medial side of the SVC and stent tube 360, to thereby force stent tube 360 against the inner wall of delivery sheath 402 and prevent stent tube 360 from deflecting towards the center of delivery sheath 402 and, accordingly, prevent the circumferentially-extending struts adjacent stent spine 910 from overlapping with stent spine 910. For example, the cylindrical spacer tube may be operatively coupled to the handle, e.g., through the sheath hub, via a thread or a catheter connected thereto, such that the cylindrical spacer tube may be retracted proximally within the proximal, delivery portion of the support upon deployment of stent 904 within the blood vessel. In some embodiments, the cylindrical spacer may be a flexible shaft.

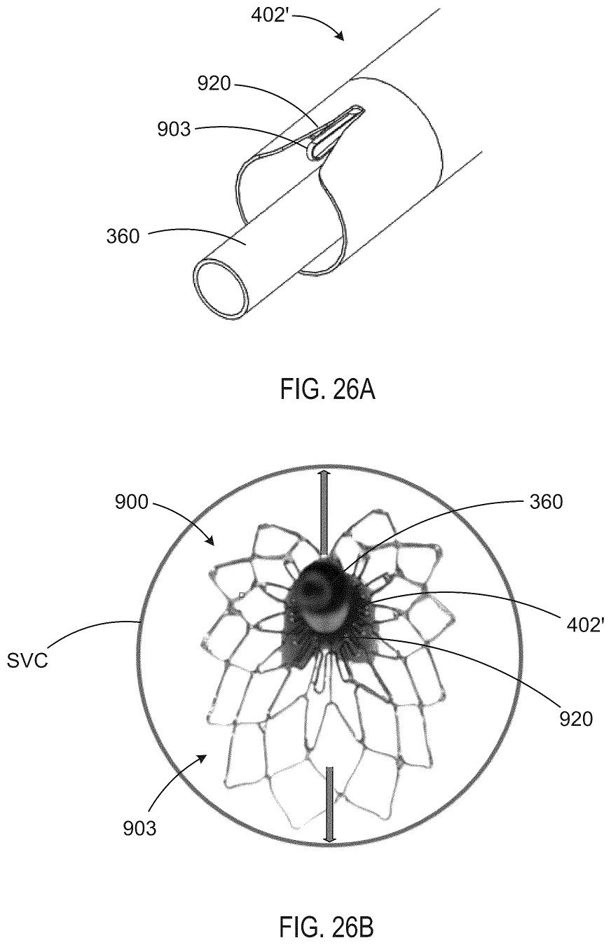

Referring now to FIGS. 26A and 26B, an exemplary delivery sheath having a slit to facilitate deployment of an expandable anchor is provided. As shown in FIG. 26A, delivery sheath 402′ may comprise slit 920 extending proximally from the distal end of delivery sheath 402′ and configured to promote deployment of stent 904 towards the medial side of the SVC more than laterally from stent tube 360. For example, slit 920 may be disposed on delivery sheath 402′ at a position opposite stent tube 360 when stent 904 is crimped in its collapsed delivery configuration within delivery sheath 402′. Accordingly, upon deployment of stent 904 from delivery sheath 402′, e.g., when slit 920 is oriented towards the medial side of the SVC, at least a portion stent 904 may be deployed through slit 920 and initially contact the medial side of the SVC, e.g., prior to the lateral sides of stent 904 contacting the lateral sides of the SVC, to thereby force delivery sheath 402′ and stent tube 360 away from the medial wall of the SVC, as shown in FIG. 26B, which results in a balance of forces during the rest of the deployment of stent 904 from delivery sheath 402′, and prevents in-folding of stent 904 during deployment.

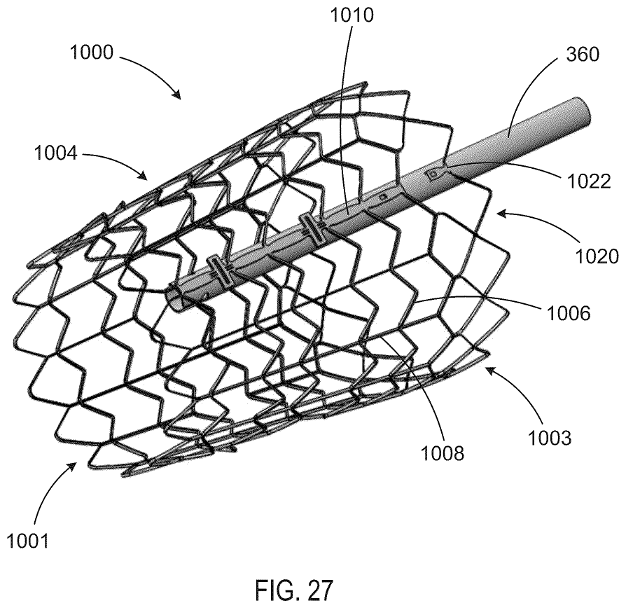

Referring now to FIG. 27, another exemplary expandable anchor having variable stiffness to facilitate deployment of the expandable anchor is provided. Anchor 1000 may comprise stent 1004, which may be constructed similar to any of the stents described herein, e.g., stents 504, 604, 604′, 604″, 704, 904, etc. For example, stent 1004 may be formed by a plurality of circumferentially-extending struts 1006, selectively interconnected via a plurality of longitudinal struts 1008, and may be coupled to stent tube 360 via stent spine 1010. The portions of circumferentially-extending struts 1006 adjacent to stent spine 1010 may be made gradually stiffer than the remaining portions of circumferentially-extending struts 1006, e.g., by gradually increasing the struts thickness or decreasing the strut length along the struts towards stent spine 1010, to thereby provide an additional resistance force to hinging of circumferentially-extending struts 1006 along stent spine 1010. In addition, as shown in FIG. 27, stent 1004 may comprise an additional circumferentially-extending strut 1020 coupled to and extending distally from distal end 1003 of stent 1004. At least a portion of circumferentially-extending strut 1020 may be coupled to stent tube 360 at a position distal to stent spine 1010, to thereby provide a more even deployment of stent 1010 from the delivery sheath and preventing in-folding of stent 1004 during deployment.

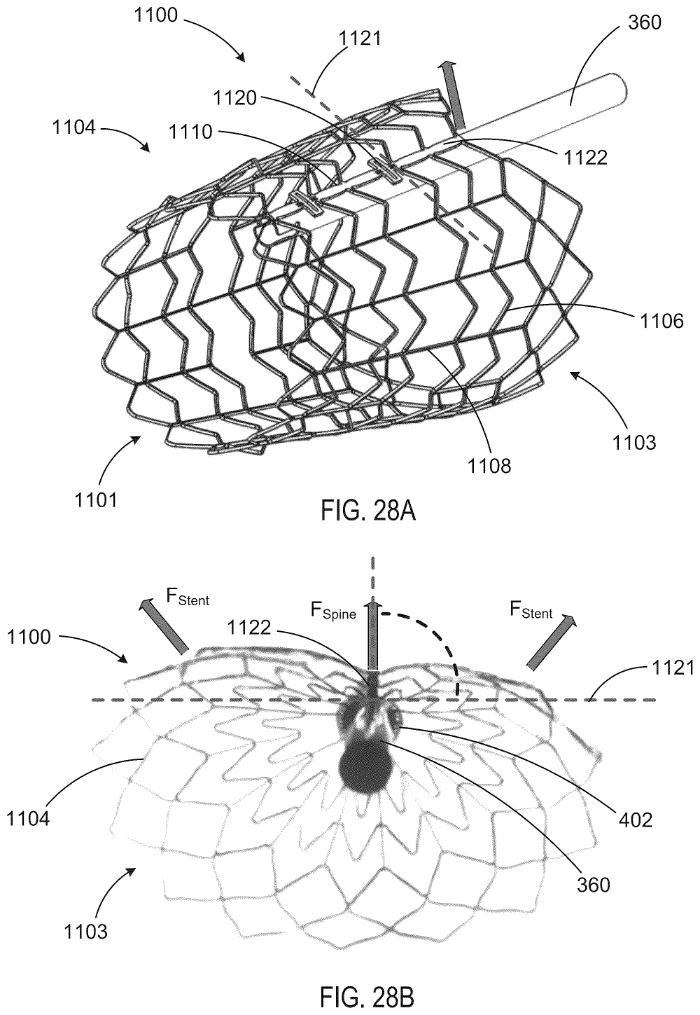

Referring now to FIGS. 28A and 28B, another exemplary expandable anchor having variable stiffness to facilitate deployment of the expandable anchor is provided. As shown in FIG. 28A, anchor 1100 may comprise stent 1104, which may be constructed similar to any of the stents described herein, e.g., stents 504, 604, 604′, 604″, 704, 904, 1004, etc. For example, stent 1104 may be formed by a plurality of circumferentially-extending struts 1106, selectively interconnected via a plurality of longitudinal struts 1108, and may be coupled to stent tube 360 via stent spine 1110. As described above, at least a portion of stent spine 1110 may be affixed to stent tube 360, e.g., at least at connection portion 1120 of stent spine 1110. Specifically, the portion of stent spine 1110 extending proximally from connection portion 1120 may be affixed to stent tube 360, either along its entire length or at additional discrete connection points along the proximal portion. The portion of stent spine 1110 extending distally from connection portion 1120, e.g., flexible stent spine portion 1122 may not be affixed to stent tube 360, such that, during deployment of stent 1104 from delivery sheath 402, radial reaction forces F_{Stent} generated by the partial deployment of stent 1104 out of delivery sheath 402 causes unconnected stent spine portion 1122 to lift radially away from stent tube 360, e.g., via F_{Spine}, as shown in FIG. 28B. For example, F_{Spine} applied to stent spine portion 1122 may cause stent spine portion 1122 to hinge about axis 1121 extending perpendicular to stent spine 1110 at connection portion 1120. As shown in FIG. 28B, lifting of stent spine portion 1122, and accordingly the portions of circumferentially-extending struts 1106 and longitudinal struts 1108 coupled thereto, results in a flatter angle at the connection between the struts and stent spine 1110, which prevents in-folding of stent 1104 during deployment. Moreover, stent spine portion 1122 may be configured to move back towards stent tube 360 upon full deployment of stent 1104, as shown in FIG. 28A.

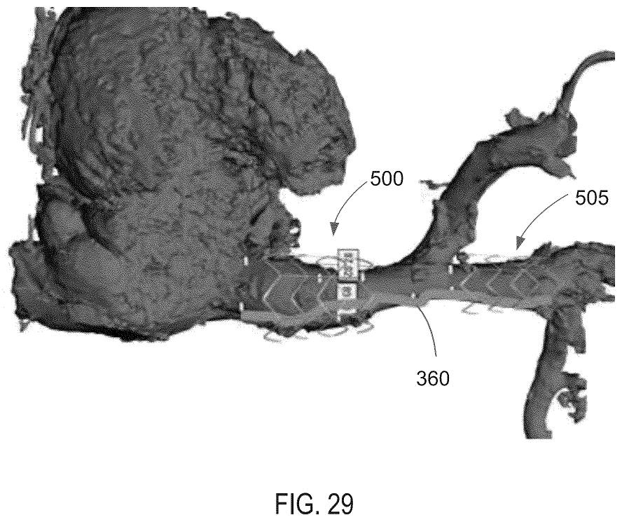

Referring now to FIG. 29, an alternative exemplary heart valve therapeutic device having two anchors is described. As shown in FIG. 29, anchor tube 360 may be coupled to anchor 500 for implantation at a first location within the patient's vasculature to support and maintain the prosthetic coaptation body (not shown) within the native heart valve. In addition, anchor tube 360 may be coupled to a second anchor 505, constructed similar to anchor 500, at a second location proximal to the first location within the patient's vasculature to provide additional support in maintaining anchor 500 at the first location and the prosthetic coaptation body within the native heart valve. Anchor 500 and second anchor 505 may have similar or different stiffness, and may exhibit the same or different radial force for anchoring in their respective locations. The anchor tube may also be shaped set so as to bias it away from the vessel wall to reduce the risk of trauma. As will be understood by a person having ordinary skill in the art, anchors 500, 505 may be replaced with any of the anchors described herein, e.g., anchors 600, 600′, 600″, 600′″, 700, 700′, 800.

The invention is not limited to the embodiments described but may be varied in construction and detail. For example, the order in which the elongated rail, the body support catheter, and the shaping catheter are disposed within each other to form the support may vary. The described embodiments are to be considered in all respects only as illustrative and not restrictive. Any part of the device may be of a material that is visible to equipment such as echo or x-ray imaging equipment. The device may further comprise a controller arranged to be implanted subcutaneously on the support to allow the position of the prosthetic coaptation body to be changed after insertion. Electromagnetic switches may be used to activate to alter the position of the distal end of the support.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

1. A device for maintaining a prosthetic heart valve device having a support at a native heart valve of a patient's heart, the device comprising:
a stent tube having a lumen configured to receive the support therethrough;
a stent coupled to the stent tube and configured to transition between a collapsed, delivery state and an expanded, deployed state to anchor the support to a blood vessel coupled to the heart such that the prosthetic heart valve device coupled to the support is positioned at the native heart valve of the patient's heart, the stent comprising a proximal region, a distal region, and a tapered profile such that a cross-sectional area of the stent increases in a direction from the proximal region towards the distal region; and
at least one antimigration element disposed on the stent and configured to resist migration of the stent in a distal direction when the stent is in the expanded, deployed state within the blood vessel.

2. The device of claim 1, wherein the stent comprises a plurality of circumferentially-extending struts selectively interconnected by a plurality of longitudinal struts.

3. The device of claim 2, wherein the plurality of circumferentially-extending struts comprises an alternating pattern of valleys and apexes, the valleys facing in a proximal direction and the apexes facing in the distal direction.

4. The device of claim 3, wherein the plurality of longitudinal struts is configured to only connect and extend between selected valleys of adjacent circumferentially-extending struts of the plurality of circumferentially-extending struts, such that the apexes of the plurality of circumferentially-extending struts are unsupported within each cell of the stent defined by a pair of adjacent circumferentially-extending struts of the plurality of circumferentially-extending struts and a pair of adjacent longitudinal struts of the plurality of longitudinal struts.

5. The device of claim 4, wherein the at least one antimigration element comprises the unsupported apexes of the plurality of circumferentially-extending struts, such that, upon radial compression of the stent, the unsupported apexes of the plurality of circumferentially-extending struts are configured to expand radially outwardly in the distal direction at a first angle relative to an outer surface of the stent to interact with an inner wall of the blood vessel and resist migration of the stent in the distal direction.

6. The device of claim 5, wherein at least one of the unsupported apexes of the plurality of circumferentially-extending struts are configured to, upon radial compression of the stent, expand radially outwardly in the distal direction at a second angle relative to the outer surface of the stent to interact with the inner wall of the blood vessel and resist migration of the stent in the distal direction, wherein the second angle is greater than the first angle.

7. The device of claim 4, wherein at least one cell of the stent comprises a local open cell geometry defined by a pair of adjacent longitudinal struts of the plurality of longitudinal struts and a pair of adjacent circumferentially-extending struts of the plurality of circumferentially-extending struts comprising a pair of valleys within the local open cell geometry that are not connected via a longitudinal strut, the local open cell geometry configured to improve flexibility and conformability of the stent within the blood vessel.

8. The device of claim 7, wherein, in the expanded, deployed state within the blood vessel, the stent comprises a bow-tie or hourglass shaped profile due to the at least one cell of the stent comprising the local open cell geometry.

9. The device of claim 3, wherein at least one longitudinal strut of the plurality of longitudinal struts is configured to extend along an entire length from a valley of a distal-most circumferentially-extending strut of the plurality of circumferentially-extending struts to a valley of a proximal-most circumferentially-extending strut of the plurality of circumferentially-extending struts, adjacent the stent tube.

10. The device of claim 2, wherein the at least one antimigration element comprises at least one friction pad coupled to at least one longitudinal strut of the plurality of longitudinal struts, and wherein distally facing edges of the at least one friction pad are configured to provide additional contact against an inner wall of the blood vessel when the stent is in the expanded, deployed state within the blood vessel to thereby resist migration of the stent in the distal direction.

11. The device of claim 10, wherein at least a portion of an outer surface of the at least one friction pad comprises a textured surface configured to provide additional friction force against the inner wall of the blood vessel when the stent is in the expanded, deployed state within the blood vessel to thereby resist migration of the stent in the distal direction.

12. The device of claim 10, wherein the at least one friction pad comprises at least one raised portion configured to be radially elevated from the at least one longitudinal strut via at least one ramped portion.

13. The device of claim 12, wherein the at least one friction pad comprises at least one opening disposed on at least one of the at least one raised portion or the at least one ramped portion, the at least one opening configured to provide additional distally facing edges configured to provide additional contact against the inner wall of the blood vessel when the stent is in the expanded, deployed state within the blood vessel to thereby resist migration of the stent in the distal direction.

14. The device of claim 12, wherein the at least one friction pad comprises a track extending along at least the at least one raised portion, the track configured to reduce impact of stiffness of the at least one longitudinal strut such that the at least one friction pad does not impact conformability of the stent to the blood vessel.

15. The device of claim 14, wherein the at least one longitudinal strut extends continuously along the at least one friction pad.

16. The device of claim 14, wherein at least one raised portion is configured to be radially elevated from the at least one longitudinal strut via a proximal ramped portion extending from a proximal flat portion extending laterally from the at least one longitudinal strut, and via a distal ramped portion extending from a distal flat portion extending laterally from the at least one longitudinal strut.

17. The device of claim 2, wherein at least one circumferentially-extending strut of the plurality of circumferentially-extending struts comprises a stiffness that increases in a direction towards a connection of the stent to the stent tube to thereby resist bending of the stent along the connection of the stent to the stent tube.

18. The device of claim 1, wherein the stent is configured to be implanted within a superior vena cava, and wherein the at least one antimigration element is disposed on the proximal region of the stent such that, when the stent is in the expanded, deployed state within the superior vena cava, the at least one antimigration element is positioned cranial to the patient's pulmonary artery and/or aorta.

19. The device of claim 1, wherein the stent is configured to be implanted within a superior vena cava, and wherein the at least one antimigration element is selectively disposed at radial positions along the stent such that, when the stent is in the expanded, deployed state within the superior vena cava, the at least one antimigration element is positioned away from the patient's aorta.

20. The device of claim 1, wherein the at least one antimigration element comprises at least one short flare configured to transition between a collapsed configuration and an expanded configuration where the at least one short flare is angled relative to the outer surface of the stent in the distal direction to interact with the inner wall of the blood vessel and resist migration of the stent in the distal direction.

21. The device of claim 1, wherein the stent comprises a proximal atraumatic portion comprising a closed cell structure extending from a proximal end of the proximal region of the stent, the proximal atraumatic portion comprising at least one thin circumferentially-extending strut that is more flexible than the plurality circumferentially-extending struts of the stent to provide a soft, gradual transition of interaction between an inner wall of the blood vessel and the stent.

22. The device of claim 21, wherein the stent is configured to be implanted within a superior vena cava, and wherein the at least one antimigration element comprises the proximal atraumatic portion such that, in the expanded, deployed state, the proximal atraumatic portion is configured to flare radially outward and conform to a confluence of the patient's left and right brachiocephalic veins coupled to the superior vena cava to resist migration of the stent in the distal direction without crossing the confluence.

23. The device of claim 1, wherein the stent comprises an expandable wire frame having variable stiffness along a length of the stent.

24. The device of claim 23, wherein the expandable wire frame comprises a decoupling section extending between and connecting a proximal-most circumferentially-extending strut of the distal region of the stent and a distal-most circumferentially-extending strut of the proximal region of the stent, the decoupling section configured to limit transmission of force from the distal region to the proximal region.

25. The device of claim 24, the decoupling section comprises a plurality of wavy struts extending between the proximal-most circumferentially-extending strut of the distal region of the stent and the distal-most circumferentially-extending strut of the proximal region of the stent.

26. The device of claim 25, wherein the plurality of wavy struts comprises an S-shape.

27. The device of claim 1, wherein the stent tube comprises at least one tilt control rib extending radially outward from an outer surface of the stent tube in a direction toward a central axis of the stent, the at least one tilt control rib sized and shaped to push the stent tube against an inner wall of a sheath when the stent is in the collapsed, delivery state within the sheath to thereby prevent in-folding of the stent during deployment from the sheath. 28 (Withdrawn) The device of claim 1, further comprising a balloon catheter configured to be disposed between the stent tube and the stent when the stent is in the collapsed, delivery state within a sheath, the balloon catheter comprising a balloon configured to be inflated during deployment of the stent from the sheath to push the stent tube away from a medial wall of the blood vessel to thereby prevent in-folding of the stent during deployment from the sheath. 29 (Original) The device of claim 1, further comprising:
a sheath comprising a slit extending proximally from a distal end of the sheath, the sheath configured to receive the stent in the collapsed, delivery state such that the stent tube is disposed oppositely from the slit, wherein, during deployment of the stent from the sheath, the slit facilitates deployment of the portion of the stent opposite the stent tube towards a medial wall of the blood vessel to thereby push the stent tube away from the medial wall of the blood vessel and prevent in-folding of the stent during deployment from the sheath.

30. The device of claim 1, wherein the stent comprises a stent spine comprising a proximal portion affixed to the stent tube and a flexible distal portion unaffixed to the stent tube, and wherein, during deployment of the stent from a sheath, the flexible distal portion of the stent spine is configured to lift radially away from the stent tube to thereby prevent in-folding of the stent during deployment from the sheath.
